(19) United States
(12) Patent Application Publication
(10) Pub. No.: US 2026/0272693 A1
(43) Pub. Date: Sep. 17, 2026

(54) Mobile Tensioning Orthosis

(71) Applicant: Jesse Matthew Lyles, Gainesville, FL (US)

(72) Inventor: Jesse Matthew Lyles, Gainesville, FL (US)

(21) Appl. No.: 19/531,621

(22) Filed: Feb. 5, 2026

Related U.S. Application Data
(60) Provisional application No. 63/772,454, filed on Mar. 15, 2025.

(51) Int. Cl. A61F 5/02

(52) U.S. Cl. CPC A61F 5/028; A61F 5/024

(57) ABSTRACT

A worn apparatus, which tensions parts of the body and optionally shifts the center point of tensioning. Planes of material are used to anchor pressure inducing assemblies which press at least two of the planes into diametrically opposite sides of the body, which pushes these sides of the body apart. Through various means the pressure induced upon the body can also be deflected, absorbed or repurposed. Maximal tensioning centers can be shifted to a desired area and an individual can wear the device for extended periods of time without muscle atrophy, while remaining mobile, without being tied to a bed, wall or weights, or reliant on sessions with a therapist. Additionally, this device may tension, through the laid out variations, all part of the spine (except the sacrum) and all appendages, up to the tips of a finger. One may tension one or more areas simultaneously.

### CROSS-REFERENCES TO RELATED APPLICATIONS

63/772,454

### STATEMENT REGARDING FEDERALLY SPONSORED RESEARCH OR DEVELOPMENT

Not Applicable

### NAMES OF PARTIES TO JOINT A JOINT RESEARCH AGREEMENT

Not Applicable

### INCORPORATION BY-REFERENCE OF MATERIAL SUBMITTED ON READ-ONLY OPTICAL DISC, AS A TEXT FILE ON AN XML FILE VIA THE PATENT ELECTRONIC SYSTEM

Not Applicable

### STATEMENT REGARDING PRIOR DISCLOSURES BY THE INVENTOR OR JOINT INVENTORS

Not Applicable

### SPECIFICATIONS

### FIELD

The present patent application relates to an orthotic device, or an orthosis, and, as such, relates to splints, braces and/or bandages; and compromises means which exerts a tensile force on the body, or a portion thereof, to stretch the body apart to a degree desired for however much time is desired.

Specifically, this invention pertains to a worn body tensioning device which transfers the force from one or more linear pressure inducing assemblies to planes which push against a body (stretching said body), without the use of weights, without the use of affixing one to a particular location by means of pulleys and weights (or otherwise), without invasively piercing the skin, without debilitating base edge induced pinpoint pressure, without constricting the body in a manner which would detrimentally atrophy muscles or restrict blood flow (if used for prolonged periods of time) with optional modifications (comprising more than two base levels, modified pressure inducing assemblies and spring base combinations) to shift the center of tensioning along the part of the body being tensioned so that, as one example, one may tension the thoracic, the lumbar, and/or the middle back.

### BACKGROUND

Tensioning of body parts goes back millennia and has predominately always employed some externally provided pulling method (traction), which requires one in traction to remain in a specific location, due to the myriad of methods and apparatuses used. These always bind one directly or indirectly (temporally or physically) to environmental objects and locations. The exceptions to date, are highly invasive procedures, which anchor hardware directly to the bones.

Tensioning is defined as the opposite of compression; the act of stretching an area. Traction technically involves some form of pulling through weights, gravity and pulleys, which tensions. All traction involves tensioning but not all tensioning needs traction.

Tensioning accomplished by this device has been done in other devices by means of pulling body parts (with weights, sometimes using pulleys); and these other devices have been appropriately called “traction devices”. Due to the unique nature of the device taught in this application (wherein it solves a lack of mobility problem and forgoes the necessity of pulleys and weights) the device is not therefore a traction device but, rather, more broadly a tensioning device, which is not a traction device.

So, although the subject of this teaching has to deal with a “tensioning” device, it's done by pushing the ends of a body apart and not by means of pulling. Indeed, it pushes parts of the body apart in a manner that allows one to freely move around and live their life, free from locational restrictions; however, the use of the label “traction device” most approximates the category under which the device would fit within the current patent classification system; because, the author could not find a body tensioning system classification of orthotics which pushed body parts apart, let alone a category which allows one non-location bound, free mobility during use of said device.

Due to the mercurial nature of language over time, there are further conflicts between highly specific underlying, textbook meanings regarding the use of the word orthoses/orthosis and “orthotic”. Vernacularly, “orthotic”, at least in the U.S, has superseded the use of the term “orthosis” (“orthoses” in the plural), and “orthotic device”. Similar to potentially missed typos regarding “tensioning” and “traction; “orthosis”, “orthotic device”, and “orthotic” might be accidentally interchangeably used, in spite of the authors best efforts to eradicate all errors from this application.

(Having dispensed with a brief reference to potential issues with terminology, the specific background of the teaching at hand will continue to be briefly explained.)

There is currently an extremely limited practice (e.g. inversion tables and gravity boots) of tensioning body parts without anchoring bolts directly into bone or externally using pulley's and weights.

Due to a deep seeded medical field zeitgeist resentments towards body stretching outside surgery (can be externally perceived as predominately seen as only accomplished through fixated traction sessions) due to the discovered complications of traction therapy, the benefits of long-run tensioning outside of surgical intervention has essentially been abandoned as feasible for decades.

Regardless, there has been good cause for the bias against tensioning. This method has fallen out of use since at least the late 1970's due to the degradation of muscles resulting from such, previously common, treatment methods, and, as such has led to a certain amount of distaste about the utility of prolonged tensioning (seen as only executable through traction).

Due to this recognition in the past, and due to tensioning being perceived as being inseparable from body traction and muscle atrophy; development of tensioning methods and the field associated with tensioning body parts through external devices has had depressed development.

As such, disc herniation and degenerating discs diseases are limited in available treatment options. Currently other options have a wide range of efficacy, invasiveness, and risks.

Currently, there are many non-invasive, TEMPORARY orthoses which only temporarily alleviate pain symptoms of conditions, but which, many times, through prolonged use, lead to other issues, such as muscle atrophy, which limits their prolonged-use utility. A group of such other specific orthoses are pliable constricting wraps (of many different styles, but typically elastic bands), commonly referred to as “back braces”.

These non-invasive, spinal orthoses may provide relief from pain; but, their use is limited due to resultant muscle atrophy from prolonged, extended use, which typically leads to further pain, and counter-productivity to certain physical therapy treatments, whose goal is the strengthening of the muscles.

Currently, when tensioning is medically directed as required, surgical intervention anchors hardware directly to the bones, as is the case with treatments using the Halo Brace. This type of bone anchoring is also currently done for height augmentation surgeries, fractured vertebrae, etc.

The area most in need of and most deprived of the use of an external, fixated, non-invasive tensioning orthoses is likely the spinal column, most predominately from the top of the thoracic down. Some past devices targeted only certain issues of the spine.

In the 19th century there were scoliosis braces. Some of these anchored upon the anterior portion of the shoulders; however these never intended, nor actually provided, total (or nigh-total) superior to inferior tensioning.

There have been some mobile orthoses developed, but they only partially laterally, and obliquely tension the back in their endeavor to laterally displace a portion of the back (similar to the cast iron scoliosis orthoses of the 19th century), all while compressing the opposite side in a manner that limits that side's muscle use, and others do similar to this.

A modern spin-off of the cast iron scoliosis brace for temporary thoracic herniation relief is the Jewlett hyperextension braces but this only partially tensions the anterior of a disc, and are only intended for temporary pain relief; definitely not full, 360 degree tensioning or 24/7 tensioning to encourage the healing of the disc ligaments through disc decompression—it merely exchanges tensioning of the anterior of a disc for compression on the opposite end, with limited force application. Prolonged use, as is the case with all current mobile braces, continues to be problematic to stabilizer muscle strength retention, due the continued-used of pliable, malleable bands for placement which constricts areas treated and untreated. Indeed, quasi-90 degrees off-set from the 19th century scoliosis brace, the Jewlett brace is ONLY used for anterior to posterior stretching.

Regarding physical therapy (and similar treatments, such as acupuncture and aquatic therapy) for long-run back pain relief, from several months of treatment, relief is fleeting in most scenarios; and it is expensive—costing hundreds of dollars per session for relief that last from mere minutes, after a session, to a few days, or more, depending on the situation. These treatments also are highly unlikely to have ANY effect on degenerating discs.

In regards to the back, chiropractic intervention is similar; it has a slightly higher success rate than other options but with an added cost and a higher likelihood of complications, verse other methods mentioned above.

Predominately, treatment of spinal discs (or other issues that might benefit from prolonged tensioning); without surgery, untethered to a location or bed, and without prolonged significant muscle atrophy; has very limited resolutions. Regarding these resolutions, invasive procedures can be performed, but these further increase the risks and potential of additional issues. These options include decompression surgeries; which essentially sand away portions of the vertebrae in order to induce a negative pressure upon a disc. Additionally, this can include anchoring to bones, with nuts and bolts, to provide constant traction to the area, or fusing the vertebral bodies. These options are not guaranteed to result in improvements, and, many times, these methods can cause more pain than was initially present.

In addition to a high chance of continued pain (if not increased pain) there is also added risks of terminal infection and life-threatening errors by the surgeon or anesthesiologists. The risks here include, but are not limited to, infection, nerve damage, excessive bleeding, blood clots, spinal instability, etc.

Other marginally successful treatments include traction tables, inversion tables, and gravity boots, which, generally require consistent; indefinite, intermittent use; and generally serve as inexpensive alternatives to constant message therapy and chiropractic interventions.

A further drawback of such devices is the up-front cost, and immobility of the function that they perform (that is the device needing to be used at a house or office). There are also space requirements within one's house or office.

Finally, the most ostensibly promising procedure is Platelet-Rich Plasma (PRP) therapy; however, QCKinetics, and others, generally relegate its use (back-wise) to the lumbar region, for the mildest cases) due to higher success rates requiring an area of low locomotion. Without proper tensioning of the disc, it is unlikely that some ligament ruptures heal much.

Furthermore QCKinectics is enduring several lawsuits, which might question the legitimacy and efficacy of the PRP procedures beyond their initially intended realm of treatments, namely the shoulders and knees/joints. PRP also costs between $1,200 and $10,000, per session; and many times it needs multiple sessions of treatment.

The closest device to address needs is the Halo Brace, which addresses a need for constant, modular and fixated tensioning; however, it requires anchoring of bolts to the skull and many sessions which use the weight of the body to set the tensioning force in place. (See the February 1968 paper, The halo. A Spinal skeletal traction fixation device By Vernon L. Nickle, Jacquelin Perry, Alice Garrett, and Malcom Heppenstall.) Indeed it set the standard for such surgical tensioning practices in lieu of older externally anchored methods of traction.

Orthotics has been a field that remains relatively slow in development. Much has not changed from molded iron casts of the ancient era, tree sap molds, and late 19th century plaster casting developments which still had considerable issues with weight and constriction of movement.

Indeed, most orthoses of the late 19th century and early 20th century mostly dealt with lateral spinal abnormalities and broken bones; and the general use of casts (from their known initial use in the archaic times with beeswax to initial plaster use in the 19th century to heal bones) took centuries, at least to become a common treatment.

Polyurethane itself was not actually produced until 1938, nor mass produced, in profitable fashion until 1956. A there continues, regarding the back, scoliosis brace developments.

The “Classic article” of the February 1989 “Symposium on Traumatic and Degenerative Lesions of the Cervical Spine” discussed additional uses of the Halo concept and led to the anchoring of hardware to the bones; and, along with issues with traction related to muscle atrophy from the mid-20th century tension methods, there has been disregard for the efficacy or even interest in alternative treatment methods to overcome non-surgical alternatives to tensioning.

The use of poly-urethane (or other such malleable, light-weight, and sturdy materials shaped with the ease of added heat like thermoplastics)) was undeveloped in the 70's, when tensioning a body fell out of use and into not only disregard but scorn; Polyurethane, produced in 1938, was not mass produced until a decade preceding the initial patent for the Halo device.

Currently polyfoam, a modern version of this, is used regularly by skilled therapists to quickly, during a visit, craft molds to aid in surgical recovery and physical therapy, but this field employs these methods and materials towards crafting exercise-prosthetics, and not tensioning devices.

Due to concerns about atrophy issues, the tensioning endeavors have solely been relegated to the use of surgical bolt-bone anchoring. As such, use of this material for tensioning braces (or for aid in producing body attached mechanical systems) is beyond the perception and perceived utility of the physical therapy field. The concepts of utility and even need, are simply out of their radar.

In the 90's, use of these thermoplastics, a type of polyfoam, in the field of orthotics was relatively new. The author read many references to studies from the early 2010's that had found the developments of use of thermoplastics lagging behind research and development of their implementation in prosthetics, and, to date, the use of a lightweight, high strength, moldable material like a low temp thermoplastic has not utilized their properties as a replacement for anchoring to the skeletal structure as wearable bases upon which hardware can be mounted or some pressure ameliorating construct used with a base which would do that.

Old norms tend to preclude competition through familiarity, and when old methodologies are broken by decades of evidence, a horn effect takes place.

This device might be another Edward Miller CDMA signal processing situation, wherein, the teaching COULD have been brought forward a 100 years earlier but procedural systemizations, and a myriad of other reasons precluded its discovery until much later than could have been done otherwise.

This teaching will deal with a device which could be made with beeswax and a thickening agent. Indeed, the claims are written as if one could just use plaster casts for the core base of the device or even metal sheets, in spite of their limitations compared to plastic alternatives.

Whatever the reason may be, a mobile device which tensions the body superiorly to inferiorly (or distally to proximally), without constriction and long run muscle atrophy, not to mention with a 360 degrees effect upon the transverse plane of a body or body part, without the use of weights, without the use of affixing one to a particular location by means of pulleys and weights (or otherwise), without invasively piercing the skin, without base edge induced pinpoint pressure, without constricting the body in a manner which would detrimentally atrophy muscles or restrict blood flow (if used for prolonged periods of time) and which allows longitudinal center of tensioning transference for use upon any part of the body short of the sacrum, skull, and distal ends of the body has yet to have been made, as of the filing of the cross-referenced provisional application: 63/772,454.

This will taught below in addition to optional modifications (comprising more than two base levels, modified pressure inducing assemblies and spring base combinations) to displace the center of tensioning along the part of the body being tensioned so that, as one example, one may tension the thoracic, the lumbar, and/or the middle back

That said, regarding a portion of that just stated endeavor, there is one potential contender regarding that which the author discovered some time after their provisional application, but that device is lacking in many capacities and functionality: Cervical Spine Brace and Traction Device, 2008 (U.S. Pat. No. 7,128,724). Yet, based upon that written, even the intentions of the cervical spine brace and traction device only tangentially, if not then marginally, approach the teachings at hand in the current application. But it is certainly a step in the right direction from that which precedes it.

Regarding Cervical Spine Brace and Traction Device, Marsh, the author, explicitly states their device is limited to use upon the neck, and it is implied, due to the functioning of the device, specifically the center of the neck.

Even in that regard the intentions of the device do not seem met. Primarily the device, over 20 years since the submission, has not lead to any products of use. Certainly nothing that would have helped the user of the device of the present application, to over-come their disc issues (albeit upon the thoracic and not neck); however in the authors early search for relief, nothing of the sort of Marsh's device was seen on the market.

Most importantly, there is an almost quarnary concern for the pinpoint pressure that would be induced by the device, at least upon the upper shoulders. Whereas the claim mention, in a quagmatic manner the existence of portions lying over the top of the shoulder, they state that these run vertically. Pictured is such, at it's minimum of third party potential understanding. There is a nigh obliqueness to the bottom of the bottom base which would still impinging upon the shoulders of a person wearing the device.

Furthermore Marsh states “lower surface of user's mandible” and again, that pictured shows a very uncomfortable lay of the base which would press into the “lower surface of the user's mandible”. It would seem very much intended that the lower base not continue anteriorly and wrap the chin (half of which mediates the lower mandible and upper mandible.) Indeed the word “surface” is used, in-line with that pictured.

In totality, there seems little, nigh of gnot, consideration given to the impingement of the bases against the user's body, which might cross into an ignorance of such inevitable issues from use. Certainly there is no intention of addressing the use of malleable materials to wrap the totality of an area at which bases press against abrupt large bodily protrusions, let alone consideration for intended, specifically designed pressure ameliorating structures and structuring. Also, that depicted would press directly into the proximal side of the user's mandible bone.

Some Velcro strips would rub a user raw shortly after donning, and there is no consideration for padding even mentioned, further enforcing the view that Marsh only borderline gnot considerate of the user's experience and endured pressure from any aspect of the device.

Indeed, regarding use, there are many more issues demonstrated by this absence of consideration in that depicted in the Cervical Spine Brace and Traction Device. A user of this device could extend and flex their neck, which would increase the pressure upon these points.

The “guides” and “small keepers”, without at least washers, seem totally in capable of nigh neck movement containment as the device seem to requisite, ostensibly not even for more than a few hours of use. Considering the device in totality is intended to be preset by therapists it is essentially defunct in its conceptualization, short of the intended goals implied and stated. The device seems to allow a flexion and extension of the neck which could compromise the integrity of the brace very quickly.

That which is written in the current application uses, many times, sometime emphatically, curvature of bases intentionally to ameliorate pressure: curvature not only at the point of large protrusions, but dependent on the chosen material (of which many are shown to be possible with a large set of options expressed), many small bodily curvatures along large areas to be tensioned. Not to mention other specific structural options, specifically for the thoracic, an armpit hammock.

Over time, with the some of the issues above, the user's neck movements will. at first, quickly loosen and displace the “guides” and “small keepers”, without a second rod per side, and eventually the neck will be able to twist more, which will put pressure on the guide pin, break it, and displace the longitudinal pressure without means to retighten.

Additionally an “acruate” collar brace would actually be what is most inferiorly pictured, and longitudinal pressure would very much press into the body or a user and constrict blood-flow.

In Dothard v. Rawlinson, 433 U.S. 321(1977 ) an author had patented a cooling box which allowed cold air down four walls of a box, but another later patent allowed for such to happen down only two wall. Because the original artist directed the constructing of the claim to require the exact structure shown in the embodiment the later artist was found to not infringe.

Indeed, it was partly due to this case, and part of the rationale behind it (a need for an artist to demonstrate their perception of obviousness) that lead the author of the application at hand to more thoroughly demonstrate all that mentioned and intended covered in their provisional claim. [That, along with learning how to write claims, the history of the why the rules are how they are, etc. led to the specifications of the art at hand, which the author feels does a much better service to community at large.]

There is, for the most basal embodiment of the current application, a distinction of a similar nature to that of this case. Marsh, limits the application of linear pressure inducing apparatuses to two lead screw actuators per sagittal side of the body, a flaw which, along with the other flaws of the “guides” and “small keepers” and such, leads to the breakdown of utility of the device.

Note that Marsh's device specifically calls for the use of one linear actuator assembly (specifically a double sided guide screw assembly, actuated by a star wheel and horizontal shaft—which may be actuated by an electric motor) per sagittal side, or more broadly, a single actuator assembly per left and right sagittal side of the body, WITHOUT actual consideration for functioning body-pinpoint pressure ameliorating modifications to the curvature of the bases, not even that of padding.

This ill-regard for negative user experience, seen by the specific placement of Velcro along areas which would abut against the skin, without any notation in the specifications for such potential need for padding needs to be addressed) The teaching at hand will resolve this intended limiting factor and introduce many pressure ameliorating structures and attachments which will help progress the art at hand towards something useful, even outside uses upon the neck, as well as provide for resolutions to other issues of functionality within Marsh's art.

Known to the author, at the time of filing, there is another, learned towards the end of finalizing their application, which merits reference and which could at least partially resolve that which has not ostensibly been resolved in the history of body tensioning endeavors. This is the Traction Apparatus and Methods (U.S. Pat. No. 10,744,024) filing in December, 2014.

However, the authors of Traction Apparatus and Methods appear to be, if this exists, a group that modifies expiring patents without actual understanding. Yet, they retain Marsh as an author and still retain “the keepers” but not their other half—the issues there in unaddressed.

They use pressure ameliorating structures like padding and even mild body shape conformity, yet they only use it upon the shoulders. This seems intended to address a shortfall of Marsh's design but stops short of demonstrating the broad understanding of intended utility of that which they are performing, intending a limit of use upon the shoulders by not addressing other areas where such would be needed.

They retain the “keepers” but give no mention to their other halves, almost as if they didn't quite understand the function of that over which they wrote. They also add ~one or more~“support member(s)” to the back of the head. These seem to be generally, adjustable, “lockable” rods, to forgo the issues with the “guides” and “small keepers”. Yet, as stated, the other half of the small keepers (that to which it connects) is missing, and issue seems to be totally ignored. It is not clear how this was intended to resolve the need for short run device stability.

Essentially this art attempts to address SOME of the issue at hand in Marsh's teachings. Yet it seems to fall considerably short of the mark of demonstrating any actual understanding of that which they uniquely contribute beyond a uniquely author bound understanding of obviousness, which preclude functionality, seemingly attempting to introduce solutions of a physical structure in a teaching of method.

Whereas “locking teeth” may be stated as an option for setting the support member's height, it is not necessarily clear if the authors are aware of what that might even broadly entail. And it continues to state use as requisite upon a therapist intermediary for all stated.

That said, the author of the application at hand sees this Traction Apparatus and Methods as an attempted step in the right direction, yet it is not quite certain what that direction (obliquely) may be?

How these lock in length in not quite clarified beyond the fact that they may incorporate teeth and such. Supposedly this is stated to notate the identified defects of Marsh's art, seemingly (to this author's eyes) without a less than glib textual demonstration of a solution beyond “locking teeth”.

There still remains a dedication to solely one linear actuator per sagittal side of the body. Considering Marsh's continued presence it would appear the intention of the padding and base folds was not necessarily for specifically pressure amelioration but rather for imitation of older halo brace variants, which rest upon the shoulder.

There are even new representation which call into question the authors'competency and intentions. For example, the hook and loop fasteners depicted in illustrations, in the new version, are both straps that jut out, both facing proximally. This is done on the top base front opening and the bottom base front opening. It's not functional without, again, no concern for the user of the device, who could twist the hook and loop at the cost of skin abrasions.

Regardless of all this, all that was claimed was a method, and it further leaves an unfulfilled need; the author of the teaching at hand will address these issues and many others to attain the intentions of this application at hand.

This is the limit of the author of the current application's knowledge of material which address the issues at hand.

The application and breadth of potential alternatives, indicating a much broader net of potential, also present due to this reality of a massive set of potential pats and combinations of those parts, will have concepts demonstrated through illustration, simply at first. A consistent style of embodiments is used below to teach the concepts necessary to resolve the shortfall in innovation regarding mobile tensioning orthoses. They will increase in complexity, introduce new concepts and art that will lead to a device which, in totality can tension most every spot of the body and pinpoint, along the longitudinal plane, areas to be targeted, and the final most complex culmination of all the teachings.

### BRIEF SUMMARY OF THE INVENTION

### GENERAL CONCEPT AND DESIGN OF A MOBILE, TENSIONING ORTHOSIS

Presented and taught is a mobile, non-invasive tensioning device, which transfers the force from one or more linear pressure inducing assemblies to planar bases (upon 2 or more base levels) which push against a body (stretching said body), allowing for the stretching of one or more body parts while also, optionally, shifting the location of the center of tensioning upon a body part: without surgical intervention and while allowing one to be unfixed to a location, without the use of weights; without the use of affixing one to a particular location by means of pulleys and weights (or otherwise); without invasively piercing the skin; without debilitating base edge induced pinpoint pressure; without constricting the body in a manner which would detrimentally atrophy muscles or restrict blood flow (if used for prolonged periods of time); with optional modifications (comprising more than two base levels, modified pressure inducing assemblies and spring base combinations) to displace the center of tensioning along the part of the body being tensioned so that, as one example, one may tension the thoracic, the lumbar, and/or the middle back. The device is most valuable in use upon non-static areas with multi-directionally moving joints—a benefit of which the author used to heal their own incurable spinal disc issues, and the stretching can be longitudinally total or partial (without matching repercussive compression opposite that part which is being partially stretched);

In its simplest form the device comprises two planes (bases for connections of tensioning hardware), which lie (partially or fully lying over a circumference of a body) over diametrically opposed areas (wholly different or partially—they may over-lap) of a body to be tensioned. The planes are pushed apart from one another through either a single linear tensioning apparatus that mediates the entirety of the circumference (a pre-compressed set of compression springs for example) or three or more linear tensioning apparatus assemblies (connected to both minimum requisite bases). The planes push into the body, which pushes the opposing ends of a body apart; and pressure pushed into the body is dissipated, absorbed or other-wise ameliorated by various means (e.g., either by shaping of the end of the planes which press into the body, attachments to the ends of the planes which press into the plane, the planes molding to contours of the body, a sort of elastic hammock with solid ends upon which rods can attach, springs, attachments molded to the contours of that into which plane would press, skin gripping mechanism, or a combination of all that).

In its most ideal form, the planar bases are composed of a material which can be easily shaped and molded to a person's specific contours, after a series of general body styles have been pre-fabricated for mass consumption. Yet, one may use Kevlar, spider silk, plaster, sheets of metal, or even 3-d printed materials combining several different types of medium (or even interwoven or laterally or longitudinally vary strips of each material). For example: displayed in one embodiment is a planar base made of bamboo sticks, shaped through steam, varied with elastic cloth between many iterations of these bamboo sticks. Depending on the material used, even if modable, one may need to fold a portion of that which is to have a carriage bolt, stick and form a thicker hole (as thermoplastics allow) or even just inserting a grommet.

Indeed, even the linear pressure inducing apparatuses have a massive breadth of potential choices available. For simplicity in the illustration, consistently used for all illustrations, to truncate a potentially massive parts list, a simple tensioning rod, as a turnbuckle with two end rods, is used. One could use segmented spindle actuators or even hydraulic cylinder actuator assemblies, ratchets and stoppers, rotary to linear pressure inducing assemblies, etc. The field of potential, pressure inducing assembly alternatives is so broad that it is mostly only referenced through examples and potentials in the claims (indeed once one starts using springs to modify the linear tensioning assemblies themselves (in a manner to displace the center of tensioning pressure or mitigate) in increasingly novel manners, the potential factorial combination based combinations leads to a number of additional embodiments which balloons further), not to mention the many different types of mounting hardware potentials and bars.

Essentially, the illustrations deal with the simplest representations of the device and the simplest modification is in line with that which is necessary to be taught and expressed (some of which can be used to divert the center of tensioning pressure anywhere used, even if limited in application within the illustrations). Upon reading case law it has become apparent that the author needs to demonstrate some level of competency regarding the breadth of their perceived intentions of obviousness; however, for illustrative representation clarity and a manageable parts list, the author sticks to simpler general embodiments in the illustration section of increasing complexity to a point of temporal and special limitation in this authors initial patent application. This choice also points towards an intended concept of modular parts for efficient mass distribution, but also leads to considerably more complex constructions.

Most directly represented in the illustrations are embodiments which tension a thoracic area of a back (a prototype used by the author); several embodiments of spring incorporated mechanism (diverting center point tensioning) used upon the thoracic embodiment for tensioning the lumbar (one of which helps the author, too); specifically molded bases for use upon the legs and neck; and a general, very simple display of a multi-area tensioning embodiment (which showcases pressure diversion (from other displayed illustrations) through the movement of bases and the ends of the linear applicators).

Of most importance is the distribution of pressure at pressure points (e.g., with cross-rods pressed against body contouring folds) and the redistribution of pressure through springs, additional base levels (of which there are a massive number of potentials demonstrated in increasing complexity).

Used for long period of times (the author used a version for almost a month) one can have constant tensioning which is extremely helpful in spinal disc ruptures; it allows them to heal. With the application in totality applied, most every disc of the spin can be targeted, which will help many, many people.

One advantage of the teaching of this application is that it provides for a device, which allows for tensioning of body parts while one is mobile and untethered to weights. It is another advantage of the present teaching that it provides for a prolonged use of such a device without accompanying muscle atrophy. It is yet another advantage of the present invention that it provides for a device that allows one to perform all daily tasks, with mild limitations, while enjoying the aforementioned benefits. Of final notation on benefits garnered, though much more can be done, one can apply this to most every body part short of the skull, sacrum and the most distal ends of the body—although, use of prosthetics at the ends is mentioned earlier for use within a future intended continuation filing.

The benefits of this device can be further coupled with other treatments such as PRP therapies in order to much more quickly heal.

The following is a summary of specific uses upon parts of the body illustrated in the drawings; however, one may use this in combination WITH current surgical procedure to tension the sacrum for example, or the tips of a finger; although the author will have a continuation filing for the use of prosthetics at the tips of digits in a later filing.

The following sections of this General Description summarize simple use of the concepts upon varying parts of the body as a lead-up to the illustrations. These simple embodiments illustrated include, thoracic tensioning embodiments, lumbar tensioning embodiments, appendage tensioning embodiment, a cervical tensioning embodiment, and a combination embodiment. Some further notes are provided throughout below for clarity and potentially needed additional information for a better understanding of the present teaching and alternative applications.

### THORACIC TENSIONING

Presented is a thoracic tensioning embodiment, which, non-surgically (barring anchoring to the bones with bolts or other hardware) anchors itself (presses upon) upon the extruding, superior and inferior portions of the torso (arm pit to the most prominent extrusion of the hips, hip bone or buttocks (or other protrusions, if present)), in order to provide mobile tensioning of the discs of the spine. Simultaneously, this embodiment allows transitioning from standing to sitting to walking, while maintaining tensioning.

This embodiment can provide (in its ideal form) 360 degrees of disc decompression from superior to inferior, which allows for negative pressure upon the nuclear pulpous (bulging or protruding through a ruptured annulus fibrosus). This allows for relief or, with time of sustained tensioning, the healing of the annulus fibrosus; OR (if not used at regimented, long periods of time) temporarily relieves pain and symptoms associated with a herniated disc, or symptoms and pain of other issues alleviated by tensioning—conditions which may, for relief, be reliant upon disc traction for relief but which require the use of previously bulky tables or bars from which to hang.

Additionally, this device may be used constantly, or periodically, for extended periods beyond an hour, allowing one to drive, walk, defecate and clean their anus, etc. while using the device.

One can begin with at least two structures. Ideally for mass production these can be approximately molded to the general contours of body type sets (either the male or female body (corpulent, ectomorphic or otherwise)). This allows for external (not within the body), pressure-resistant anchoring points upon which one can exert inferior to superior pressure (in order to apply tensioning to the spinal column while simultaneously, through this pressure, increasing the distance between the inferior and superior, at least semi-planar base(s) of the orthotic device).

In an embodiment of the thoracic version of this device (pictured and noted below) a portion of the planar base, just under the armpits, is folded, rounded and padded. Inflatable bladders may also be placed here for additional comfort, increasing the distribution of pressure upon the body from the planar-base-pressure-inducing rods, or other similar linear pressure inducing actuator assemblies; however there are many other options. For example one may craft a flexible cradle for the armpit, lined with pressure absorbent material, with hard surfaces on the anterior and posterior of the arm and back which connections through rods at multiple angles, to the immediately inferior base nearby, distributing pressure that way.

Additionally, two turnbuckles are attached just under, posteriorly and anteriorly to each arm pit, and other ends of the lower portion of the turnbuckles are anchored to a lower planar base, lying over the protrusions of the hips and buttocks. Although one may use a plethora of intermediary bases (base levels) and modified tensioning assemblies to specifically target an area along the back.

Thus, by tightening the turnbuckles, pressure is gradually applied to the hips and arm pits, tensioning the spine, specifically the thoracic, but dependent on the size of the planar bases and location of the ends of the tensioning apparatus assemblies.

The most prevalent thoracic embodiment is a drawn rendition of an initial prototype used by the author of the teaching at hand which demonstrates proportions which would most directly tension T 5-8 if needed, but changes in the inferior anchoring point of the tensioning assemblies can shift this center point. One could also change the size of the bases or number of base levels used, or modify tensioning hardware used (see claims) to adjust the area of the back at which the back tensioning happens.

The turn buckle ends are rounded and moderately affixed in place with nuts, washers, and bolts, allowing one to move/swing the upper portion out of the way (anteriorly or posteriorly) to, for example, clean themselves after defecating.

In one variant of the thoracic embodiment, a portion of the planar base abutting against the arm pit is folded in a round manner across and over a rounded, straight cross-rod. This rounded rod may be present in non-thoracic targeted embodiments of this device and may curve, to, in general, follow the contours of a generally cylindrical portion of the body up to the entire circumference of the body. A bolt is passed through these cross-rods and planar bases; and this bolt is the same bolt (in the embodiments) which affixes the tensioning rods/turnbuckles.

In an embodiment this cross-rod is a turnbuckle, and in another embodiment this cross-rod is a flat cross-bar.

In an embodiment of the device, used for the thoracic discs, there are two separate planar superior planar bases, one for each arm pit, allowing for more air-flow and breath-ability and the aforementioned swing-ability.

In an embodiment there is a single disunion of the lower planar base for donning and doffing the device. In an embodiment there are hook and loop fasteners used to apply pressure to the lower portion of the planar base, which secures the lower planar base better to the body contours, and, if desired creates greater pressure against the skin (using the natural gripping of the skin) for increased pressure upon which the tensioning works. Hook and loop fasteners are used to hold the opening closed. In another embodiments other types of buckles and straps are used, for example, ladder straps and ratcheting buckles, as one might find on a snowboard. One may also just simply use an elastic strap.

Due to the at least semi-rigid nature of the planar bases, muscle atrophy is avoided through prolonged use due to the stabilizer muscles not being constricted!

In an embodiment there are hook and loop fasteners placed upon the two superior planar bases for holding up the device (acting as shoulder straps). Specifically, these shoulder straps may be used to hold the device up while one fastens the lower planar bases in place. Assuming an opening is chosen for the lower planar base, the straps would hold the device up while lower hook and loop fasteners (or a general brace or other tightening methods) are used.

In another embodiment of the device, flat bars, with spaced holes, are used in lieu of turnbuckles. Two couples of flat bars are used for each superior anchoring point (arm pits) and each couple of flat bars partially overlap its other mate. To tension, the overlap is then shortened for each couple increasing the total length the two mated, longitudinally running, flat bars—the overlap being affixed for the desired total length, creating the desired distance/pressure, ideally, prior to donning the device or while one optionally, hangs. Though this is at the outskirts of the totality of that to be taught. Indeed, predominately this teaching was motivated by a desire to get away from external object and person dependent tension level setting. However, shown here is an option at that borderline.

In an embodiment, handles are placed upon the inferior planar bases by cutting and folding the planar base material upon cross-bars, similar to that done upon the upper two planar bases of the thoracic embodiment device. These folds can be used while donning the device to better affix the bottom planar base to the desired spot.

In the predominately depicted thoracic embodiment of the device, two superior planar bases are used, one under (and abutting against) each armpit at rounded, padded edges of the armpit abutting portions of the upper planar bases. There are shoulder straps and lateral straps for holding the top planar bases together, and the lower planar base has an opening at the front, held closed by straps, when worn.

In this thoracic embodiment of the device, turnbuckles are used just anteriorly and posteriorly to each armpit and attach directly beneath, to the lower planar base.

In this embodiment, turnbuckles twist and increase in length to tensioning of the back. Additionally, the lower edge of the inferior planar base is flanged away from the thighs. This is done to prevent the turnbuckle induced pressure from pressing the lower planar base into the thighs, while a user is seated, in a method that would otherwise constrict blood flow.

### LUMBAR TENSIONING

In the predominately depicted embodiment of the lumbar tensioning embodiment, one begins with a version of the thoracic tensioning embodiment in which there are two superior planar bases, the inferior support planar base, tensioning rods (turnbuckles) and cross-rods.

With these planar bases, springs are used to concentrate/divert the tensioning pressure of the thoracic embodiment towards the lower back.

In this embodiment, two new levels of planar bases (two planar bases per side/two planar bases per superior planar base) are introduced between the superior and inferior planar bases. On each side, one of these new planar bases is inferior to the other new planar base, creating an inferior middle base and superior middle base on each side of the body.

The inferior portion of the tensioning rods remain connected to the original inferior planar base; however the superior portion of these tensioning rods attach to the inferior of an inferior middle bases (the base which is immediately superior to the inferior planar base connections): two tensioning rods per middle base, per side.

The inferior middle bases are rounded or squared (aware from the body being tensioned) at their tops. The superior middle base has a squared superior portion (away from the body being tensioned), into which the superior portion of the inferior middle bases fit, or inserts.

In this embodiment the inferior portion (opposite the side of the plane from which the squared opening bends) of each of the superior middle bases attaches to the inferior portion of one of the superior most bases in a manner that allows it to move in the manner that a hinge might allow, the two pieces flat against one another at one side of a complete inferior to superior swing.

Also, a double coil torsion spring provides resistance for the superior middle base to rotate in a manner that its superior portion is as superior as the original superior planar base, when rotated. When the springs are engaged, the planar bases (superior middle and superior) abut against each along the exterior plane of the superior planar base, lying flat against one another.

Inside the fold of the superior middle base is a compression spring, or set of springs, or spring like mechanisms, which further resists the superior to inferior pressure resulting from the tightening of the turnbuckles (or otherwise). Note that the opening of the superior middle base faces inferiorly when in this position, completing a full superior swing.

The entirety of the structure is created such that, on each side the superior of the inferior middle base inserts into the gap (squared opening) of the superior middle base. Furthermore, on each side, the inferior middle base presses against the compression spring(s) therein. This pressure also actuates the double coil torsion spring, as the superior middle bases swing up and are engaged by the lower middle base through the pressure exerted by tensioning rods.

To reiterate, when nigh-full pressure is applied, the superior middle base lies flat against the inferior of the original superior planar base, with the opening of the inferior middle bases facing inferiorly, the protrusions distal to the that side which lies against its respective superior planar base.

This modification to the thoracic tensioning orthosis is one of several variations which directs the center of the tensioning rod pressure towards the lower back, from the thoracic region. Indeed, depending on the center of tensioning of a general core body tensioning variant, one can invert the longitudinal side upon which the springs are used or use spring like mechanism inside a plethora of linear tensioning assemblies to specifically target. Other variants devised from the principles on display can place pressure along most disc within the spine and most runs of a body part suitable for use of the device.

Some other embodiments, specifically involving spring-base combinations, are demonstrated in the detailed description of the drawings below.

Generally, in the lumbar tensioning variants (seen in many different methods of employment in the detailed descriptions), springs and/or multiple additional levels of planar bases are used in many different manners to displace the tensioning pressure of the thoracic embodiments, upon the lower back and lumbar region; however, as stated, center tensioning pressure can be done through varying placements of springs, bases, and changing tensioning apparatuses to vary the local of the center of tensioning in order to specifically tension individual discs, if some disc needs additional pressure than a general area tensioning provides. (Examples are given in claims the most complex verbalized.)

Many variants of the lumbar tensioning embodiment exist below and may be combined with many other methods laid out in this teaching and the concepts may be used on other parts of the body.

### APPENDAGE TENSIONING

In an appendage embodiment, in accordance with the concept of thoracic tensioning (and that of the general embodiment), there are two planar bases that lie over the circumference of an appendage, one distal planar base to another planar proximal base.

In an embodiment these planar bases match the general protrusions of an appendage; yet, end attachments specifically molded, or with springs absorbing force, etc., may also be done.

In an embodiment, the distal planar base conforms to the top of the heel of a foot and the dorsal side of the foot, an anchor point to resist pressure which would push the distal planar base in a distal direction. Also, a proximal planar base conforms to the top of the area of the lower leg just at the bottom and beneath the femur and thigh and is molded such that the proximal end of the proximal planar bases resists pressure to the proximal planar base in the proximal direction. The proximal edge of this proximal planar base may mold to the knee's protrusion on one side and the lower rear thigh (when flexed) on the other posterior side.

Various padding or air bladders may be used upon the proximal edge of the proximal planar base to improve the anchoring of the proximal planar base against the distal edge of the knee and/or the lateral protrusion of the lower thigh.

In an embodiment tensioning hardware, which elongates, runs the longitudinal length of the portion being tensioned. The proximal ends attach to the proximal planar base and the distal ends attach to the distal planar base.

In an embodiment, as the tensioning rods elongate, the two planar bases separate in distance and (directly or indirectly) press against the points of the body which resist this pressure, this action tensions the area between the planar bases, without surgically anchoring to the bones.

As is, the embodiments used and implied herein are sufficient for use upon the upper arm, lower arm, upper thigh, etc.

### CERVICAL TENSIONING

In another embodiment the neck may be tensioned using the same methodologies of the thoracic tensioning embodiments; however, (most effectively using thermoplastics) an inferior planar base is molded to the upper shoulders and lower neck and another planar base is molded to the area from the jaw to the occipital bulb, the circumference of the lower skull.

Tensioning as is illustrated as a couple of turn buckle assemblies on the left and right side of the body (with half in front and half in back). The ends of these turnbuckles are attached by cross-rods which cross the circumference of the body, with each end of a cross-rod attached to a place at which a turnbuckle attaches; a loop of cross-rods for the superior ends of the tensioning rods and a loop of cross-rods for the inferior end of the tensioning rods.

Engagement of the device is accomplished through the turning of the turnbuckles.

The cervical embodiment serves to separate the discs of the cervical spine while allowing mild cervical rotation, and may not be useful in some cases, wherein complete immobility is needed. It does provide a non-constrictive tensioning of the neck, mitigating muscle atrophy as cervical herniated discs heal, in line with the concepts of the general embodiment.

### COMBINATION TENSIONING

The general conceptualization here, is that of an embodiment which, utilizing the concepts earlier, induces the tensioning hardware effects upon multiple areas of a body simultaneously or positions additional planar bases (akin to positioning tensioning rods upon a planar base in different positions) on new planar base levels to divert the center of tensioning pressure.

In an embodiment, this is done with multiple tensioning hardware connected levels of bases.

In an embodiment, inferiorly attached to an embodiment of the cervical tensioning embodiment, there is a base level which lies over the full circumference of the torso just under the arm pits and presses against large breast or a large belly of the upper torso. Pictured is a reused variant of a lower planar base used in other embodiment illustrations.

This embodiment allows, after a tensioning is set upon the cervical discs, additional pressure to be induced from a lower spot, thus lowering the center at which tensioning happens, towards a spot of the back that would be unreachable in other, previous, specific embodiments.

There are a considerable number of complicated modifications that can be made to increase the utility and feasibility of this variant, which illustrated and displayed as is for a more simplistic illustration and parts list; however many of these methods are mentioned in the claims and many times used in conjunction with one another to further aid in recreation of this device and its many possible variants.

This simple illustration also displays the use of modularity in a set of planar bases, that be prefixed to varying body types. Hence, depicted is a reuse of a lower planar base with straps holding open an opening.

### BRIEF DESCRIPTION OF THE SEVERAL VIEWS OF THE DRAWINGS

In the following portion of the present description, the teachings for this patent application will be explained in more detail, with reference to the illustrated embodiments shown in the drawings, in which:

FIG. 1 is a three dimensional view of a basal embodiment, using the minimum concepts, with bases molded to an unidentified general body part.

FIG. 2 is an axial sliced view of the basal embodiment, the angle through the top center opening to the bottom, which omits the body being tensioned.

FIG. 3 is an axial sliced view of the basal embodiment, the angle through the bottom center opening to the top, which omits the body being tensioned.

FIG. 4 is a three dimensional, generally-body-part-contoured (oriented top to bottom, as if used in-use) view of the embodiment of the thoracic-tensioning embodiment of the device.

FIG. 5 is a view of FIG. 4, slightly rotated left (viewer perspective) along the transverse plane.

FIG. 6 is a view based upon FIG. 5 but shows FIG. 4 instead rotated towards the right (along the transverse plane).

FIG. 7 is FIG. 4, with the opening to the left of the viewer's frame of reference.

FIG. 8 is a view centered on the back of that which is displayed in FIG. 4-7, showing the backside of the device.

FIG. 9 is a representation of that depicted in FIG. 8 but with the superior two-part planar base leaning out to show the internal side of FIG. 4's front, not yet seen.

FIG. 10 is a representation in line with FIG. 9 but with more distortion of the superior planar bases, showing more of the inside.

FIG. 11 is a representation of an alternate thoracic tensioning embodiment, represented in FIG. 4-10, and only shows the device. The images show the curved planes (of FIG. 4-10) as flat and cuts off the bolt fastening materials, for ease of demonstrating alternative hardware.

FIG. 12 is a view of FIG. 11 if the view were rotated 90 degrees upwards along the y axis of the viewer's frame of reference (upward from the displayed bottom); it displays fastening materials used and doesn't include the alternative lower planar base. Put another way, it shows a flattened version of an embodiment of FIG. 4-10, seen in FIG. 11; it shows alternative fastening materials used (both displayed in the general embodiment) but not an alternative lower planar base.

FIG. 13 is similar to FIG. 12 but is a view which has the lower planar base rotated 90 degrees in the opposite direction as that rotation to produce FIG. 12. Similarly, it includes the fastening materials from the previous image.

FIG. 14 is a blowout of one superior planar base (left or right) of FIG. 4-10 in which the top planar base is a general top planar base, and which shows the manner in which the hardware, in these illustrations are connected.

FIG. 15 is a three dimensional, in-use-oriented (generally-body-part-contoured) view of an embodiment of the device for Lumbar/lower back tensioning.

FIG. 16 is an embodiment based upon FIG. 15, with a different manner of force/tensioning placement mechanization shown.

FIG. 17 is an embodiment based upon FIG. 16, with a different manner of force/tensioning placement mechanization shown.

FIG. 18 is a lumbar tensioning embodiment based upon FIG. 17, with a different manner of force/tensioning placement mechanization shown.

FIG. 19 is a blow-out of the uniquely distinct aspects of FIG. 18, the concept of construction, and displays a spring mechanism used in a lumbar tensioning embodiment.

FIG. 20 is a representation of FIG. 15 with the rounded planar bases flattened out, to show a front-on, outside-the-body-being-tensioned view of the embodiment. Similar to FIG. 11, the image shows the curved planes as flat but doesn't cut off the fastening material. (This does NOT flatten the spring housing base, for clarity sake.)

FIG. 21 is a representation of FIG. 20 rotated 90 degreed along the x axis, either direction, from the viewer's perspective, of FIG. 20. (The spring abutting mold and the spring housing base are not flattened, in order to demonstrate the spacing and how the device engages the springs.) The bottom is to the left and the top is to the right.

FIG. 22 is a representation of FIG. 20 rotated 90 degrees along the y axis, bottom towards the top, from the viewer's perspective, showing the bottom up of the device. (The spring housing is not flattened and the compression springs are omitted for ease of viewing.)

FIG. 23 is a profile/side view of FIG. 16; the body contouring wrap is flattened out.

FIG. 24 is a profile/side view of FIG. 15; the body contouring wrap is flattened out.

FIG. 25 is a profile/side view of FIG. 17; the body contouring wrap is flattened out.

FIG. 26 is a profile/side view of FIG. 18, another lumbar tensioning embodiment; the body contouring wrap is flattened out.

FIG. 27 is a profile/side view of a new embodiment for tensioning the lower back, presented in a similar manner as FIG. 23-26.

FIG. 28 is blow-out of FIG. 15, which shows only the user's right side of the device above the inferior planar base, for clarity and simplicity; the two upper sides mirror each other.

FIG. 29 is blow-out of that depicted in FIG. 27, alternatively only showing the mirrored user perspective left side of the device, blown out, above the lower planar base.

FIG. 30 is a three dimensional, in-use (generally-body-part-contoured) view of a specific use (upon two lower legs), of the basal concepts verse a general representation in FIG. 1, used upon the each lower leg of a person (pictured are two identical versions of the device upon both lower legs of a human).

FIG. 31 is a representation in line with FIGS. 2 and 3, showing a superior to inferior view of one side of FIG. 30. The view is axial slice between the proximal and distal planar bases of one of the two identical devices of FIG. 30, looking distally, and the images omits non-device matter, for clarity.

FIG. 32 is a three-dimensional, in-use (generally-body-part-contoured) view of a specific use (upon the cervical spine) of the basal concepts verse a general representation in in FIG. 1, using a quasi-profile general representation of a human body from the chest up.

FIG. 33 is an alternate view of FIG. 32, rotated about 45 degrees from the left shoulder towards the previous center of the chest.

FIG. 34 is an embodiment of a combination tensioning device discussed in “combination tensioning” subsection of the “brief summary of the invention” section, above.

### LEGEND

101. Object to be tensioned
102. Large turnbuckle end rod (“s”/hook ending)
103. Bolt holes
104. Rounded cross-rod (which is curved to a length which is a quarter of the object around which it lies)
105. Large Turnbuckle (part of a turnbuckle assembly)
106. Washer
107. Squared Nut (Hex or similar) (with or without nylon)
108. Carriage bolt
109. Top/superior/proximal planar base wrap/opposing planar base (illustratively a top planar base in General)
110. Bottom/inferior/distal planar base/opposing planar base (illustratively a bottom planar base in General)
111. Flat cross-bar
112. Wing nut
113. Hook strap
114. Loop strap
115. Folded portion of a planar base which can act as a handle or cover cross-bars/rods.
116. Left superior planar base (compliment or right superior planar base on the same base level)
117. Right superior planar base (compliment or right superior planar base on the same base level)
118. Flange
119. double coil torsion spring
120. Small turn buckle
121. Small end rod (eye ending)
122. cross-rod (as a cylindrical rod-eye ending)
123. Foam Padding
124. Gel padding
125. Elastic band
126. Large turnbuckle end rod with jaw ending
127. Flat bar with holes (allowing for adjustable tensioning)
128. Variant planar base (made of alternating or interwoven bands of rigid and flexible material).
129. Ratchet fastener
130. Ladder strap
131. Stitch-closure buckle
132. Stitch-closure strap
133. Compression spring
134. Rivet Fastener
135. Compression spring housing
136. Flat, rectangular bar (for use with springs)
137. Guide pin rod
138. Generally cubed nut/hardware (in which two opposing sides have a threaded hole, one for a guide pin rod and one for a small screw with a flat head)
139. Flat planar superior middle base, with its inferior edge folded into a “u” shape (into which another planar base plane may snugly slide
140. Leaf spring
141. Brad fastener
142. Pliable flat, squared material such as cloth.
143. Spacer (length and width made to its use where referenced in the drawings).
144. Chicago screw sleeve nut
145. Chicago screw mating bolt
146. Inverted-“j” shape inferior middle base
147. Scissor Rod
148. First Alternate compression spring housing folded in a “u” shape on one end
149. Channel rod (prismatic)
150. Cap for channel rod
151. “u” shaped base/bar, with a width of shaped to width of an intermediary base
152. Flat, unfolded intermediary base (middle base level and inferior middle base level)
153. Compression spring rod
154. First alternate, compression spring resisting inferior middle base
155. Second alternate superior compression spring housing
156. Second alternate, compression spring resisting, inferior middle base (alternate inferior middle base)
157. Proximal base wrap for appendage
158. Distal base for wrap for appendage
159. Occipital bun
160. Superior cervical base mold
161. Inferior cervical base mold
162. Small flat-head fasteners
163. Hinge
164. Telescopic guide pin
165. Cylindrical rod with protruding nodule
166. Holes for rivets and screws/sex bolts

### DETAILED DESCRIPTION OF THE ILLUSTRATIONS

In the following detailed description, the device; for this patent application, in the form of a mobile tensioning orthosis and mobile, tensioning orthosis; will be described by the illustrated embodiments, and their representations in the drawings. These illustrations, simple in their devisement for clarity and concision, shall display concepts for the formulation of several other intended variants.

For simplicity, the planar bases are illustrated as semi-body conforming, derived from a series of pre-made constructs for a wide variety of body types, and made out of a re-moldable material for more personalized modifications of the end user; however, it should be noted, that one could very easily use Kevlar, hard plastic sheets, or planar sheets of metal as a bases upon which to attach hardware or to deliver or divert the pressure of a wide range of linear pressure inducing actuator assemblies. In which case further modifications would simply require the attachment of pressure diverting attachments and structures to the edges of these base which would press against the body for tensioning. Some alternative are alluded to in examples in the claims section; however display and illustration of such parts (some very complex) would create a much more complex set of illustrations, parts list, and descriptions (ref general description for more).

Suffice it to say that it is the opinion of the author that the following embodiments follow an ideal material use and composition for a mass-manufactured product (with a large of array of general body shape preset planar bases) that can be optionally tailored to a person's unique form or parts thereof.

For ease of understanding the General, appendage and cervical embodiments; they will be described as if the user has specially molded the planar bases exactly to their unique form. Even though the general embodiment is upon an undefined body part (representative of a general application), the views show moldable curves in FIG. 2 and FIG. 3.

Further note that displacement of the center of tensioning can be accomplished by simply changing the location to which a linear tensioning apparatus (illustrated as turnbuckle assemblies) attaches to the bases, adding more bases (discussed in the combination embodiment), or the use of springs (with new spring-based components resisting bases, but also with springs within linear tensioning apparatuses, which, due to succinctness issues in an already long specifications, are referenced briefly in the claims for one skilled in the art to better understand these alternative center of tensioning diverting method options).

### General Basic Tensioning Embodiment

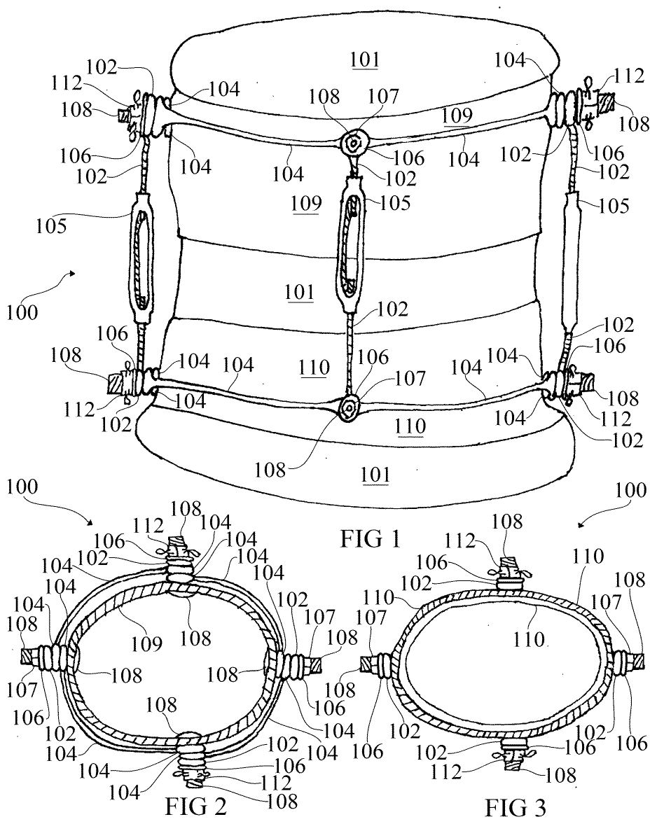

A first embodiment, a general embodiment of a tensioning orthotic device 100 is illustrated in FIGS. 1, 2 and 3. These figures show The General Orthosis 100. This comprises an object to be tensioned 101, large turnbuckles 105 and large turnbuckle end rod 102, carriage bolts 108, washers 106, nuts (wing nuts 112, or hex nuts 107), a single top/superior/proximal planar base wrap 109, a bottom/inferior/distal planar base 110 and curved, rounded cross-rods 104.

The single top/superior/proximal planar base wrap 109 is attached to the contrary bottom/inferior/distal planar base wrap 110 by use of tensioning rods (large turnbuckles 105 with large turnbuckle end rods 102 on each of their longitudinally running ends)—the large turnbuckle end rods 102 attach to either the top/superior/proximal planar base 109 or the bottom/inferior/distal planar base 110 depending upon which end of the body-longitudinally-running large turnbuckles 105 they attach. (The large turnbuckles 105 are equally distanced along the general circumference of the object being tensioned—in the figures (FIG. 1-3) there are four large turnbuckles 105 used.)

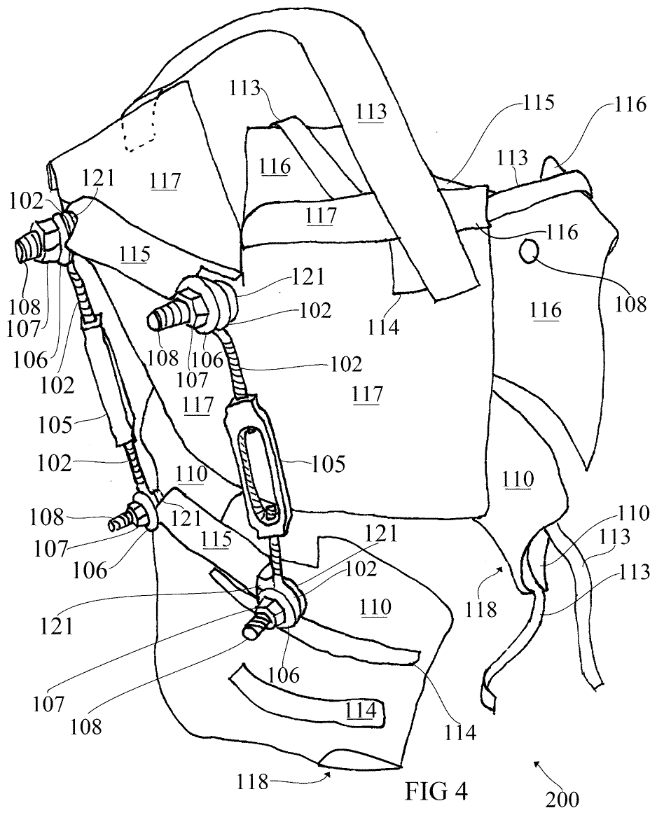

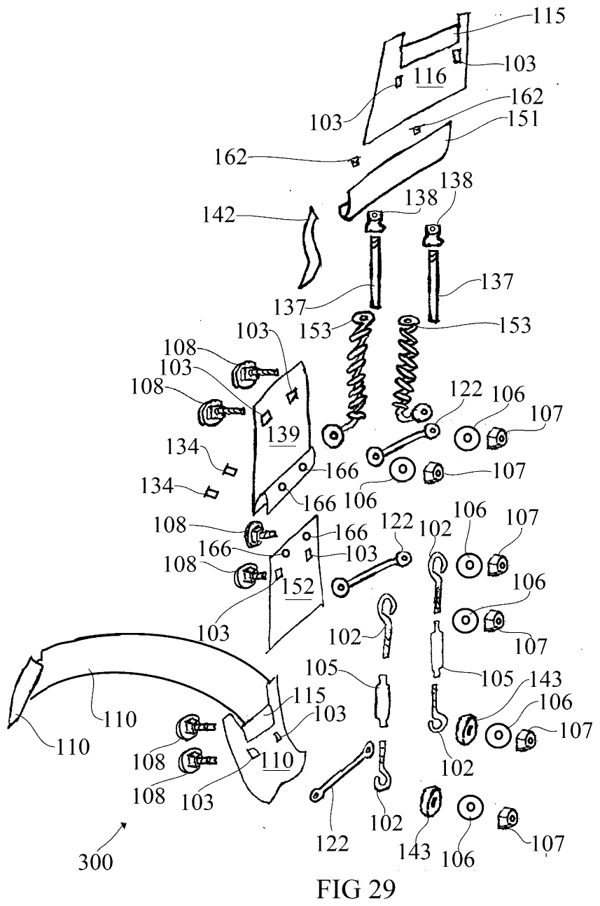

In later, more complicated embodiments (demonstrated in the other images (FIG. 4 FIG. 29) the top/superior/proximal planar base wrap 109 is split into a superior left planar base 116 and superior right planar base 117 partitions.

These planar bases (bottom/inferior planar base 110, top/superior/proximal planar base wrap 109 ((later) superior left planar base 116, and (later) superior right planar base 117)) in most embodiments, are lightweight, rigid and sturdy, made out of a low temperature thermoplastic or similarly functioning material. However, the material used may not necessarily need to be thermosplastic, so long as the material is generally sturdy and lightweight and able to at least generally mold to the contours of the body. Such material could easily be 3-d printed or even made of a composite of different cuts of material.

These planar bases (bottom/inferior/distal planar base 110 and top superior/proximal planar base 109) lie around the circumference of a body, potentially partially molded to variances in curvature—their ends opposite their contrary planar base are intended to abut against at least mildly prominent protrusions of the body to be tensioned 101, for maximal effectiveness; however, these protrusions may not need be so prominent, because the addition of pressure ameliorating attachments, waxes, and padding may be enough to give the desired amount of tensioning, as the large turnbuckles 105 loosen their connection to their attached large turnbuckle end rods 102.

FIG. 2 shows this general tensioning embodiment 100 from the top down, through the center of the planar bases which lies around the circumference of a body; FIG. 3 shows this general tensioning embodiment 100 from the bottom up through the center. In FIGS. 2 and 3 omitted is the object to be tensioned 101, for clarity.

Notice that in FIG. 3 the arrow line attached to the bottom/inferior planar base 110 (from inside the elliptical object) touches a non-hashed area, but (outside this a line for an inferior planar base touches a hashed object. This indicates that the wrap bends inwards at the bottom. The hash marked and non-hash marked circles are both identified as the bottom/inferior planar base 110 for this reason—to illustratively show optional body curvature conformity of a planar base to mild protrusions/curvatures of a body.

Further illustrated in this basic general embodiment, the ends of the top/superior/proximal planar base 109 and bottom/inferior planar base 110 are shaped such that they conform to general protrusions of an approximately cylinder-like objects (such as a torso or appendage), thus providing resistance to pressure created by the lengthening of the large turnbuckle end rods 102 as the large turnbuckles 105 twist; however other more complicated methods of ameliorating pinpoint pressure at protrusions can performed and attachments which distribute this pressure in the same means of a base molding to the curvatures. This resistance and linear pressure increases the distance between the bottom/inferior planar base 110 and top/superior/proximal planar base/wrap 109, thus tensioning an area at the middle of the object to be tensioned 101.

In this embodiment, the large turnbuckle end rods 102 are attached to the top/superior/proximal planar base/wrap 109 and bottom/inferior/distal 110 planar bases by use of carriage bolts 108, washers 106 and hex nuts 107 (or alternatively displayed wing nuts 112); however, other means of fastening may be employed, e.g., nylon bolts, glues, push-pins, rivets, and Chicago screws.

To very generally describe the art at hand, depicted in FIG. 1-3 is the most simplistic, basal representation of the concepts behind the art at hand. Two diametrically opposed planar bases (a single top/superior/proximal planar base/wrap 109 is attached to a contrary bottom/inferior/distal planar base wrap 110) are wrapped around a body to be tensioned 101, pressing, as the tensioning rod pressure is applied, against general protrusions on contrary ends of a body to be tensioned's 101, longitudinally contrary ends.

Longitudinally opposed planar bases are connected by tensioning rods (large turnbuckle end rods 102 on each side of longitudinally running large turnbuckles 105—a series of which is equally spaced along the axial circumference of the body to be tensioned 101).

As the longitudinal distance of the two large turnbuckle end rods 102 per large turnbuckle 105 combos increases the distance between the planar bases is pushed apart, which press against ends of the body to be tensioned 101. The resistance on each end to this longitudinal pressure stretches or tensions the body to be tensioned 101.

Whereas this can be easily applied to areas with broad protrusions free of pressure points, which could be damaged with prolonged pressure, some additionally considerations changes need to be made for application to other parts of the body, namely the thoracic.

The predominate distinct between this general illustration and that of the thoracic device is the pressure reduction methods used at the points at which the bases would press into the body and significantly cut of blood flow. The simplest means illustrated are shaping the ends to distribute the pinpoint pressure more broadly upon the area at hand, properly position rods and cross bars, and using various types of padding. This most simple solution is illustrated; however, there is an array of much better methods described below, such as longer extensions upon the molds to the pinpoint areas, flanges, spring use, attachments which cradle the armpit in a flexible mesh, using smaller contours of the body with additional bases a mixture of this, etc.

The next illustrations, that of a thoracic variant device, will display the simplest means for ease of clarity and due to fiscal, temporal, and artistic limitations of the author. It is interesting to note the paramount importance of diverting pressure upon joints and blood vessels. Indeed, the more complicated versions (beyond the general, such as the thoracic variant) could have be constructed with plaster molds (or even archaic beeswax molds) with the pinpoint pressure mitigating solutions provided herein, such as body molded spring attachment peripherals and sufficiently spongy padding. Yet, this has not happened.

The rest of this teaching will demonstrate specific, unique additions to the basal concepts of this teaching upon the entirety of the back (minus the sacrum).

It should be noted that the author of this application has ONLY been saved from debilitating/disabling back pain because of their creation of this device and some of its embodiments, when over 13 doctors and specialist said there was nothing else to be done—when it was said the author's back issues would get worse!

### Thoracic Tensioning Embodiments

### First Embodiment of a Thoracic Tensioning Orthosis 200

In the following section, application of the core methods applied in the general embodiment 100 are used and modified to create embodiments which specifically tension the thoracic spine, without the need for invasive surgery. Considerations have been given to blood flow, pressure points, and past issues of prolonged orthosis use, which will become apparent as the embodiments and their pictured images are shown and described below.

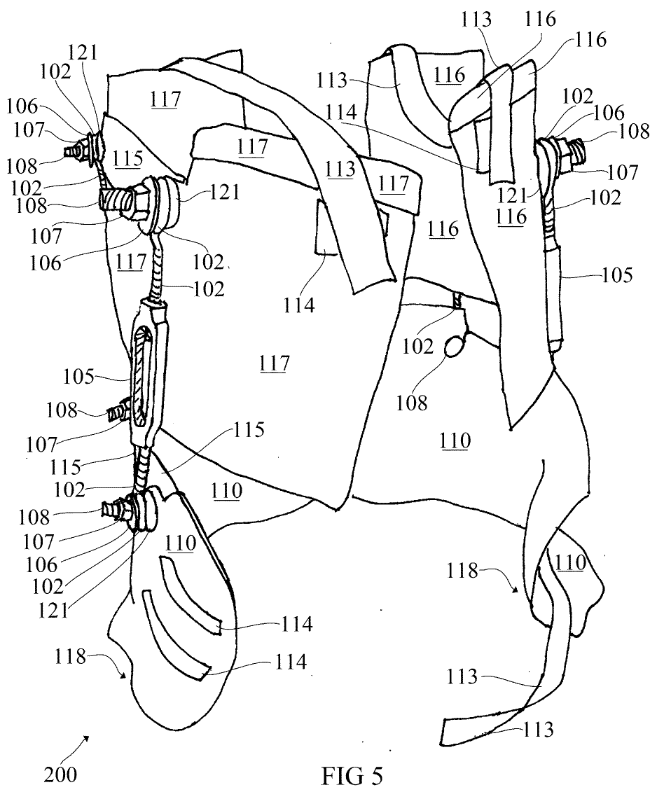

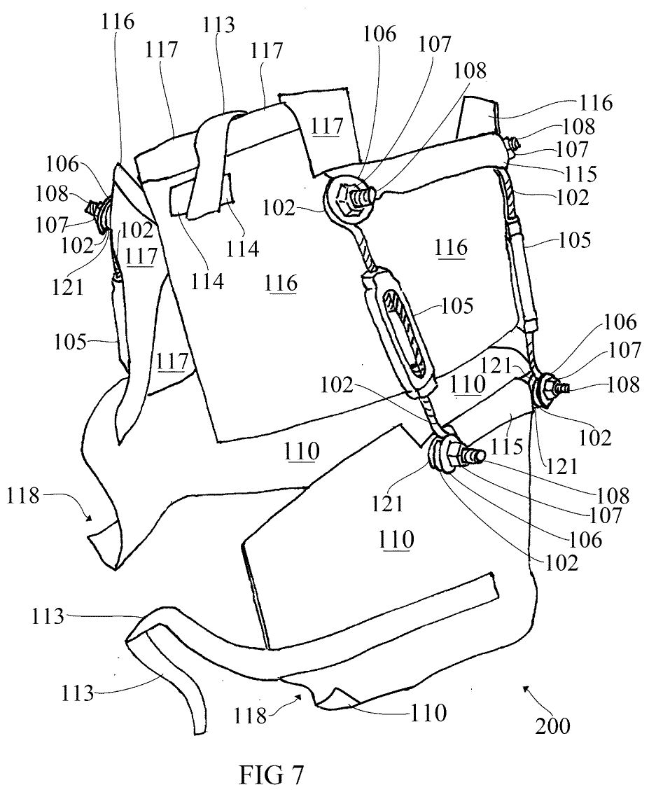

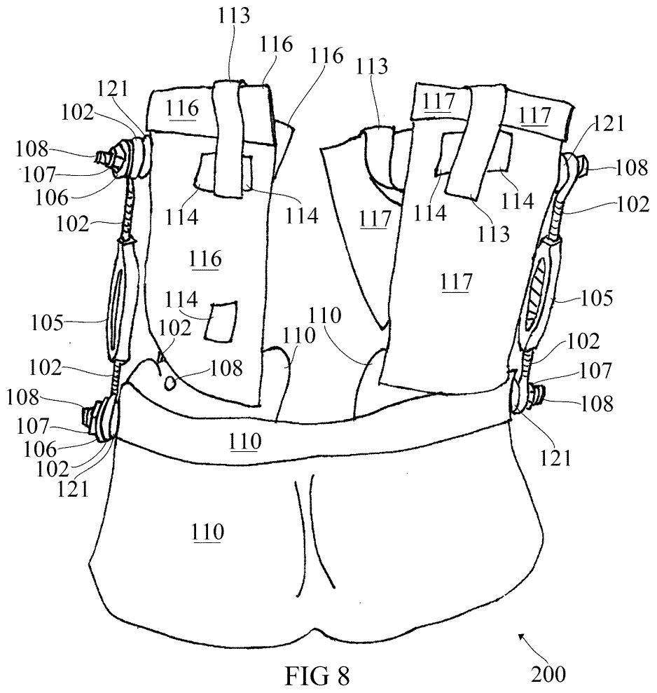

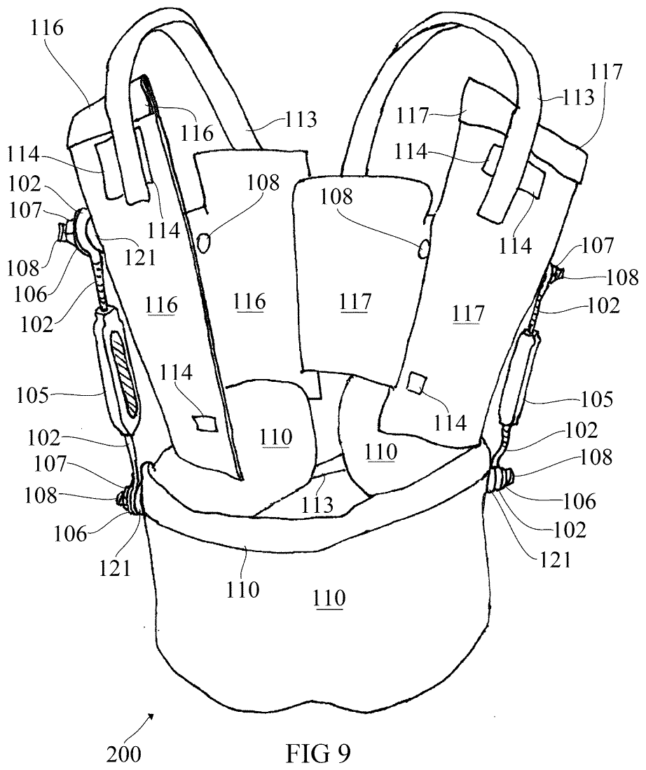

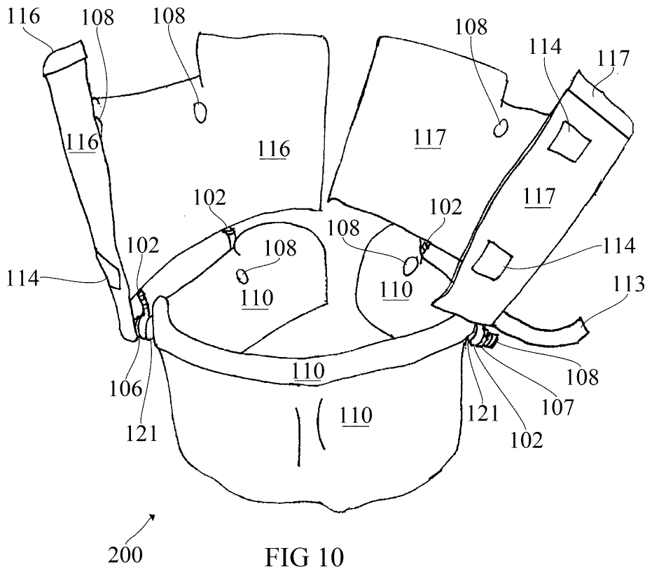

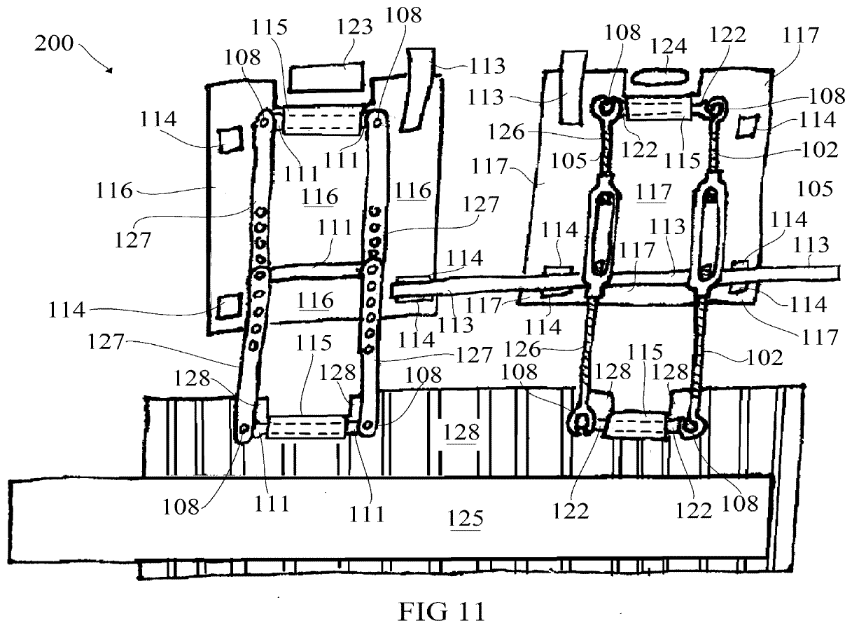

The second embodiment presented is the first embodiment of a thoracic tensioning orthotic 200 device, presented in FIG. 4-10. Additional modifications and alternatives of construction are shown in a second, third, and forth embodiment of a thoracic tensioning orthosis 200, seen in FIG. 11-13. The breadth of variants here begins a slow increase in complexity to aid in the presentation of increasingly complicated structures, depending on the audience that might use this literature.

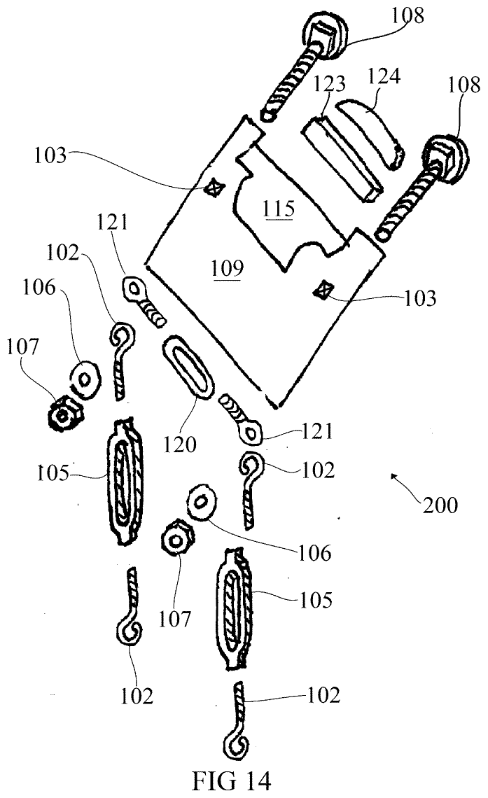

FIG. 14 shows a blowout of one side of a non-specific top planar base wrap 109, presented as if one side (left or right) of either the general 100 or thoracic 200 tensioning device embodiment. A general representation of the top wrap is used because the hardware structuring is essentially the same. This depiction is done to show the location of hardware, and to best demonstrate the connections of the tensioning rods to pressure inducing planar bases. The planar base in the blow out is flattened, without body conforming curves shown, for ease of understanding.

In the first embodiment of a thoracic tensioning orthosis 200, the inferior planar base 110 is not a single, unbroken planar base, for conceptual demonstration purposes, as with the general embodiment 100, but, rather, opened at one end for ease of donning and doffing.

The first embodiment of a thoracic tensioning orthotic 200 is essentially the same as the general embodiment 100. The predominate differences are that only hex nuts 107 are used, the device is used upon the torso, the tensioning hardware is positioned such that one longitudinally running large turnbuckle 105 is posterior and anterior to each armpit, handle folds 115 (to aid in pressure deflection or to aid in positioning the device) exist in addition to pressure diverting flanges 118.

In this embodiment, shown in FIG. 4-10 hook 113 and loop 114 fasteners are used for shoulder straps for ease of donning, and could be inverted from that which is shown—hook 113 used in place of loop 114.

Shown in FIGS. 4, 5, 6 and 7 are optional loop fasteners 114 on the inferior planar base 110, wherein tightening of the inferior planar base may be provided, post donning, by use of the hook 113 fastener portion of the shoulder straps. Coupled with a loop 114 fastener, affixed to the inferior planar base 110, a user can hold the planar base in place and ensure better resistance to the pressure created by the large turnbuckles 105, in addition to creating increased surface area contact with skin, further resisting such pressure.

Due to the rigid nature of the inferior planar base 110, muscle atrophy from prolonged use is reduced, and a tight connection of the inferior planar base's 110 opening may be maintained for extremely long periods.

In this embodiment, the top/superior/proximal planar base/wrap 109, from the general embodiment 100, is split into two separate planar bases (superior left planar base 116 and superior right planar base 117). These both have a cut and fold 115 made to act as a handle and to distribute the pressure exerted by the turnbuckles, which is applied to the arm pits and core body abutting portion of the upper arm.

In this embodiment, another cut and fold 115 is made on the inferior planar base 110, beneath that made on superior left 116 and superior right 117 planar bases. These act as handles for ease of donning and doffing, or for modular use higher up on the torso. These second, inferior cut and folds 115, however, are optional, as seen in later embodiments, wherein they are absent for demonstrative clarity.

Note that the superior right planar base 117 attaches to the right side (user perspective) of the inferior planar base 110 by means of longitudinally running large turn buckles 105, which are (inferior to superior) in the center of two large turnbuckle end rods 102 (which make the direct connection to the top and bottom planar bases by means of carriage bolts 108, washers 106 and hex nuts 107. The same is done upon the left side of the device.

In this embodiment, similar to the general embodiment of the tensioning orthotic device 100, large turnbuckles 105 and large turnbuckle end rods 102, carriage bolts 108, washers 106, and hex nuts 107. However, contrary to the first embodiment, cross bracing, for stability of the structure and pressure distribution, is provided by small turnbuckles 120 (for ease of construction, without the need to prefab varying lengths) and small turn buckle end rod 121. These mostly pass under the handles/cut and folds 115, but ideally should press along the inside of the fold.

There are four small turnbuckles 120, under each fold 115. These each attach to lateral small turnbuckle end rods 121 on each of their sides. The non-small turnbuckle 120 attached ends of these small turnbuckle end rods 121 rest their opening against the openings of the local (left to right) large turnbuckle end rod 102 holes and these ends share a carriage bolt 108, which affixes, through washer 106 and hex nuts 107, the larger turnbuckle end rod 102 to their relevant planar bases, as in the general embodiment 100.

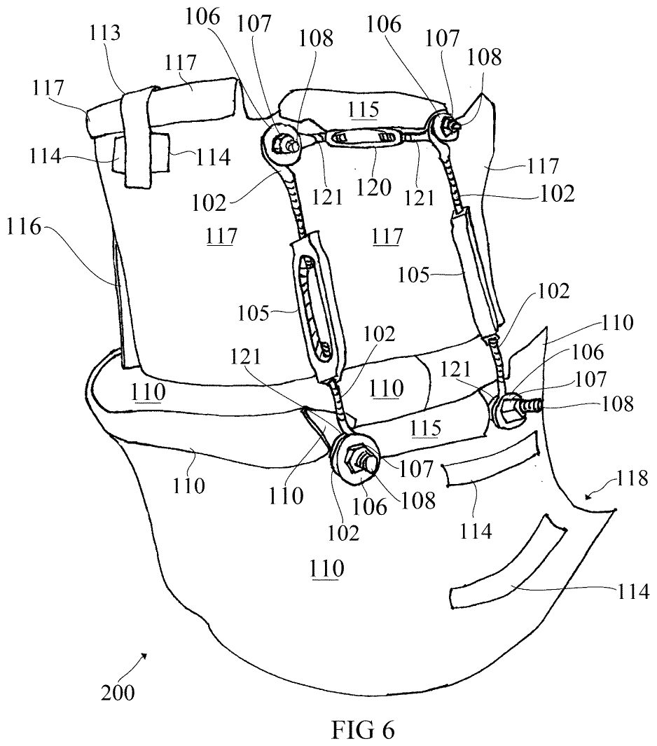

This is most clearly seen in a depiction of this embodiment in the top of FIG. 6. Although, a blow-out of the general thoracic embodiment 200 much more clearly demonstrates this and it's relations to the connection of all the parts, which is pictured in FIG. 14.

In this embodiment the large turnbuckles 105 are positioned so that their length lies upon the length of the body to be tensioned 101, and generally beneath the arm pits. They are also positioned so that twisting the large turnbuckles 105 produces elongation and pressure from inferior to superior and vice versa. Two rest in this manner on each side of the torso, beneath each arm, and each couple is positioned so that one end of the superior of the large turnbuckle 105 is positioned on opposite ends of the arms along the sagittal plane, one posterior to each arm pit and one anterior to each arm pit.

In this embodiment the large turnbuckle end rods 102 are attached to the large turnbuckles 105, and the unthreaded sides of the large turnbuckle end rods 102 are abutted against the unthreaded end of the small turnbuckle end rods 121, which, itself, has it's threaded end attached to the small turnbuckle 120, itself positioned under the handle fold 115 of either the superior left planar base 116 or superior right planar base 117.

In this embodiment, the unthreaded ends of the small turnbuckle end rod 121 (eye endings), through which the long-threaded side of a carriage bolt 108 may pass. Similarly, the unthreaded end of the large turnbuckle end rod 102. Yet, a carriage bolt's stalk may also pass through.

In this embodiment, squared bolt holes 103 are cut into the three planar bases (superior left planar base 116, superior right planar base 117, and inferior planar base 110) and positioned at the points of the overlaps of the small turnbuckle end rod 121 and large turnbuckle end rods'102 overlaps.

These carriage bolt holes 103 provides resistance for the carriage bolt when a hex nut 107, along with a washer 106, is affixed to the threaded ends, which holds the hardware in place, in lieu of anchoring to bones.

In this embodiment there is also a flange 118 (mild, partial, curved fold), at the inferior opening of the inferior planar base 110. This diverts pressure from a singular point and or plane, and prevents blood vessel constriction when one is seated.

In this embodiment the inferior planar base is brought together and better abutted against the body by the aforementioned hook 113 and loop 114 fasteners and/or and additional elastic brace.

All these pieces affixed together, as described above, create a device pictured in FIG. 4-10, this embodiment.

After one dons this embodiment, one simply needs to twist the large turn buckles to provide tensioning to the thoracic spine, having resistance provided by a general body conforming base, armpit protrusions, hip protrusions and (if needed) skin contact.

Additionally, padding may be used. For example, padding at the superior handle folds 115 works to a degree, but padding against the portions of the skin abutting planes is also effective at reducing the pressure needed at the armpit. Further complexity could involve the use of elastic type bands (or some interweaving of thermoplastics and elastic) between two hard bases which lie against the posterior and exterior the arm, forming a sort of meshed cradle, connection at varying angles through rods between the superior base and this cradle. Indeed spring between the base and the armpit could attach to many different forms and further absorb pressure.

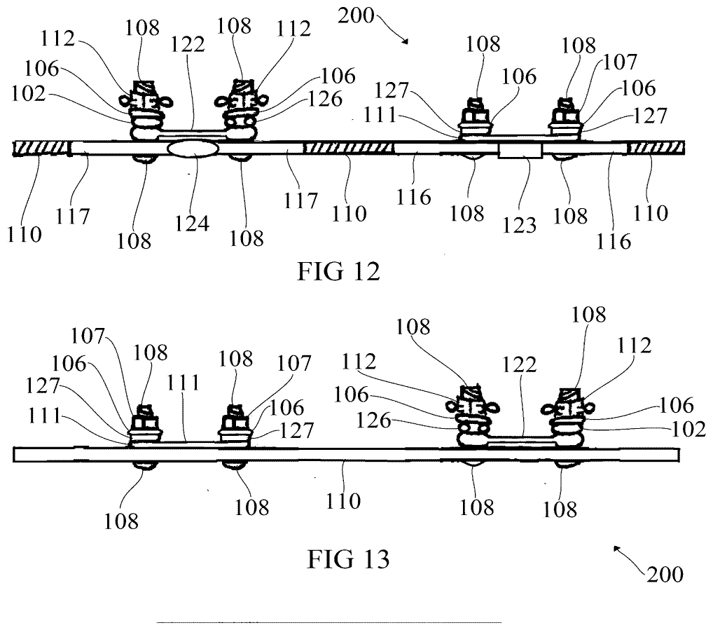

Due to the need for clarity within the images, the glued padding has been excluded from all depictions and the armpit padding has been excluded from FIG. 4-10, but are shown in the second embodiment of a thoracic tensioning orthosis 200, as gel padding 124 and foam padding 123, in FIGS. 11 and 12.

It is recommended to combine the thoracic device variant with PRP treatment sessions. The use of the device, however, is not limited to extended use, and may also be used for temporary pain relief than one might normally get by the use of a traction or inversion table, but without the need for the space to store such devices or the costs to procure them.

If one needs to clean themselves after defecating then one need merely to slightly loosen the hex nut 107, and swing either superior planar base (superior left planar base 116 or superior right planar base 117) out of the way, so that the hands may reach. (One could also just temporarily shorten the large turnbuckle end rods 102 on one side of the device.)

Furthermore, additional personal user preferences can involve tensioning pressure adjustments (for example, as one moves from a seated position to a standing position) may be made by adjusting the large turnbuckles 105, with stops for each position, if a doctor desires a specific amount of force, or, as seen in the claims, a modified tensioning rod (housing a spring), that can twist to disengage and then twist to re-tighten).

As referenced earlier, although low temp thermoplastic casting material is applied here, and used with a prototype, it is not necessary for thermoplastic or specific body molds to be used for the device to be used effectively.

Note that the base material need not be molded to specific body contours but may be generally molded to body types and gender types, due to the hip protrusions and armpits being the predominate points of anchoring against the force of the turnbuckles, regarding the thoracic illustrations. But as stated, one could use any material shaped to cylindrical, so long as pressure reduction is accomplished at the points of pressure when the bases are pushed apart as mentioned above and more thoroughly (though not exhaustively) displayed in claim 1.

This further does not preclude the use of other unique curves of a typical body composition, of a high-enough frequency to have a large numbers of preset molds made to generally common torso shapes and sizes, for manufacture and sale, for example, prefabricated planar base shapes for groups of the population, which could be specifically males with large distended bellies, or who have other types of unique protrusions in sufficient frequency within the population, to have a stock of preset general molds on hand.

Additionally, using low temperature thermoplastic also allows users to make individual mild modifications on a personal treatment level, but one could also simply 3-d print simple light weight rigid polymer planar bases only generally shaped to the circumferential lengths, attaching various pressure ameliorating structures to the ends which would abut against body protrusions.

In later embodiments, for demonstration, other types of fasteners are used and may be interchangeable. This is a simple demonstration of the simplest level at which an elephantine number of alternative embodiments becomes apparent. For simplicity of representation these are pointed out here and more complicated alternative constructions are described in the claims.

Fastener alternatives, used to assemble the device, include, but are not limited to, rivets fasteners 134, sex bolts/Chicago screws (Chicago screw sleeve nut 144 and Chicago screw mating bolt 145), wing nuts 112, etc. Alternative types of strap fasteners (for donning the device and holding it tightly in place) such as an elastic band 125, ratchet fastener/buckle 129 and ladder straps 130, and stitch-closure buckle 131 and stitch-closure straps 132, etc, can be used.

A blow-out of the hardware connections at the top (either side) is in FIG. 14, and is a general representation which only shows the hardware and general representation of a general top/superior/proximal planar base wrap 109, to more succinctly express verse displaying a left and right superior base 116/117 re-used in other illustrations for clarity purposes and indications of modularity.

The depiction, in FIG. 14, varied in specific parts used, (for example an omission of other bases) is solely used to depict how the main hardware could be assembled in the other, similar, embodiments.

It includes two carriage bolts 108, gel padding 124, foam padding 123, handle fold 115, two carriage bolt holes 103, two small turnbuckle end rod 121, one small turnbuckle 120, four large turnbuckle end rod 102, two washers 106, two hex nuts 107, and two large turn buckles 105, all of which would predominately represent the construction of the superior planar base of one side of a general representation of the device, previously described.

The lay out of the image demonstrates the manner in which the carriage bolts 108 pass through the rest of the hardware, and demonstrate the location of the carriage bolt holes 103, not viewable in many images presented of any thoracic embodiment 200, nor in the general embodiment 100; and it serves as an example of this type of hardware's assemblage in, even the cervical 500, and appendage 400 embodiments displayed below.

### Second Embodiment of a Thoracic Tensioning Orthosis 200

A second embodiment of a thoracic tensioning orthosis 200 is shown in FIG. 11, a flat view (contours of bases and curves of planes flattened,); and it is similar in most every capacity to the immediately preceding embodiment, (seen in FIG. 4-10) except for the demonstration of a few alterations.

These alterations include the use of overlapping, longitudinally run, flat bars with holes 127 and two flat cross-bars 111 on the left side (simple alternatives previously illustrated turnbuckle assemblies and cross-bars). Additionally, there is an alternate inferior planar base 110 as variant planar base 128.

Finally, there is another alternation made upon one of the other large turnbuckles end rods. The tensioning rod on the posterior right (center right) side, in place of the large turn buckle end rods 102 are alternate end rods called Large turnbuckle end rod with jaw ending 126. The tensioning rod on the anterior right remains the same. Also displayed is optional armpit padding as foam padding 123 and gel padding 124.

There is also an elastic band 125, similar to a standard back brace, affixed (e.g., glued or sowed) to a variant planar base 128, which is composed of alternating bands of rigid and flexible material (such as bamboo) and elastic mesh.

In this embodiment, alternative tensioning and cross-bar hardware is used and shown by leaving off any nuts that might affix to the carriage bolts 108.

Previously demonstrated, large turnbuckles 105 and large turnbuckle end rods 102 are used under the arms, with one being placed just posterior and one just anterior to where each arm would lie. Three of these four positions in this embodiment, display simple alternates to simple tensioning rods that could be used.

In this embodiment (and as alternates in place of large turnbuckles 105 (and their associated large turnbuckle end rods 102), used upon the left side, upon both hitherto end rod connection points) are two couples of longitudinally positioned, long, flat bars with holes 127, with two of each couple overlapping its counterpart of the couple at their center terminal ends (one of the couple above the other). These alternate bars replace the two large turnbuckle 105 tensioning end rods of the superior left planar base 116, and are a slow introduction to increasingly complicated alternative possible.

These long, flat bars with holes 127 have a single hole on one polar end of their length, the end not overlapping. A carriage bolt 108 passes through at the standard point, at which the ends of the replaced large turnbuckle rods attached to the planar bases.

On the other end of these long flat bars with holes 127, the portion upon which the long, flat bars with holes 127 overlap, is a run of equally spaced holes, which allow for the length of the two overlapped rods to be adjusted.

In this embodiment, used as crossbars, in place of the small turnbuckles 120 and small turnbuckle end rod 121, are two smaller flat cross-bars 111, with a hole at each polar end of length, through which a carriage bolt 108 passes.

In this embodiment, a further alternative cross-bar is used upon the right side of the inferior planar base 110, and the right superior planar base 117, (also under the respective cut and fold 115/handle). In this instance, this further alternative cross-bar is a single, cylindrical cross-rod 122, with circular ends through which a carriage bolt 108 passes. The ends of this cylindrical cross-rod 122 is similar to the small turnbuckle end rod's 121 unthreaded end, eye shaped.

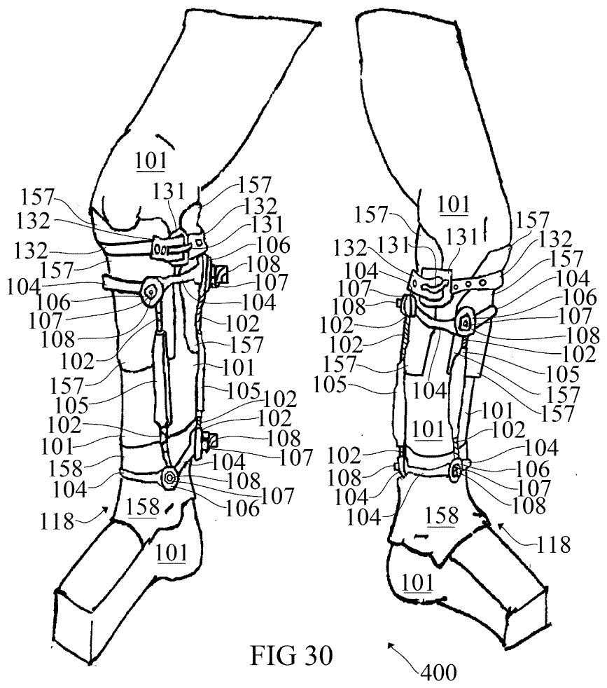

This alternative cross-bar (cylindrical cross-rod 122) is used in all of the following images besides FIG. 30-33.

Finally, in this embodiment, an alternative to a large turn buckle end rod 102, an alternative large turnbuckle end rod with jaw ending 126 is shown. An original large turn buckle end rod 102 is replaced by a turnbuckle bolt with a “u” shaped ending (an alternative Large turnbuckle end rod with jaw ending 126); and this is shown on the left/center side of the right superior planar base 117. The originally used large turnbuckle end rods 102 are still present to the right of this, in this depiction, for comparison.

In this embodiment, the hook fastener 113 is still used as a shoulder strap, but is also used, naturally, along with loop fastener 114, as a lateral strap for the superior left planar base 116 and superior right planar base 117, holding them closer together and toward the body, and is depicted in line with the latter use. The most superior hook fasteners 113 point up, with reduced depicted length, for space considerations and clarity of depiction.

Finally, in this embodiment a gel padding 124 is used above the superior right planar base's 117 handle fold 115 and foam padding 123 is similarly used on the opposite side.

The construction of the device is otherwise identical to the first embodiment of the thoracic tensioning orthosis variant 200 of the general/conceptual tensioning device 100.

### Third Embodiment of a Thoracic Tensioning Orthosis 200

A third embodiment of the thoracic tensioning device 200 is essentially identical to the second embodiment and excludes the inferior planar bases made of alternating rigid and mesh material (the variant planar base 128) and the elastic band 125 there upon it, using instead the originally presented solid inferior planar base 110. This is seen in FIG. 12, a top-down profile view (the bases flatted (not curved to a body) and FIG. 13, the opposite angle, bottom to top.

The hook 113 and loop 114 fasteners upon the superior right planar base 117 and superior left planar base 116 are also omitted due to the confusion their depiction might add if put upon the depictions shown, at the angles shown in FIGS. 12 and 13. They are presumed, for functionality of the device, present in this embodiment.

For clarity, handle folds 115 on the superior right planar base 117 and superior left planar base 116 are omitted to show top down (FIG. 12) and bottom up (FIG. 13) view of the different types of hardware used in the second embodiment, from a new angel, but with aforementioned illustrative alterations. Additionally, foam padding 123 and gel padding 124 are used where the point's handle folds 115 would be.

That is to say, FIGS. 12 and 13, depict the third embodiment, an essential replica of the second embodiment, showing a profile of the flat, in-representation, seen in FIG. 11, with modifications as stated.

These modifications are for demonstration of alternative constructions and to better display a growing set of increasingly complex alternatives that can be use. To further develop a growing understanding of the growing complexity of that which will lead to a stated eventuality in the general description. This clarity is provided to demonstrate the intended scope of coverage embodied in this teaching for the thoracic embodiments 200, and in totality--to hint at further complexity to come, ultimately leading up to the claims that will be specified shortly.

In this embodiment, the inferior planar base 110, is the same as the inferior planar base 110 used in the first thoracic embodiment; however the alternative rods (from the second thoracic embodiment) are used and placed as they were in the second embodiment, to provide clarity on how they would fit together and appear from a view along the flat profile of the planar bases (superior left planar base 116, right superior planar base 117 and inferior base 110). Additionally, as in FIG. 1, hex nuts 107 AND wing nuts 112 are used.

This embodiment is assembled and used in the same manner as the immediately preceding second thoracic embodiment 200, and uses the remaining pieces pictured at the new angle: carriage bolts 108, washers 106, cylindrical cross-rod 122, inferior planar base 110, right superior planar base 117, superior left planar base 116, flat cross-bar 111, long flat bars with holes 127 (for tensioning), the handle fold 115, large turnbuckle end rods 102, and alternate Large turnbuckle end rod with jaw ending (126).

Loosening the large turnbuckle end rods 102 (or alternate Large turnbuckle end rods with jaw ending 126) connections to their associated large turnbuckles 105 induces pressure upon the superior right planar base 117 and induces pressure upon the body contours against which the planar bases abut, thus tensioning the body on the side planar base separation. (Increases in the length of the long flat bars with holes (127) overlaps would match this on the other side.)

### Note

The carriage bolt holes 103, retained in an illustrative reuse of the superior bases (superior left planar base 116 and superior right planar base 117), for clarity and succinctness purposes, which are/would be visible are still depicted and addressed in the text, in order to be in accordance with the rules governing patent applications.

Moving in order of decreasing simplicity, the next set variants (illustrated lumbar tensioning devices), begin to use springs and more than two lateral levels of planar bases to displace pressure, much more complicated applications of the principles employed in the next section are laid out in the claims.

Although, as illustrated, these variants specifically tension the lumbar region—or lower back in general (depending on the size of the bases and the positioning of the linear pressure inducing assemblies)—variations to size and longitudinal placement can be used to tension many different areas of the back or many different areas of another body part more directly.

### Lumbar Tensioning Embodiments 300

A third variation, of the general embodiment for tensioning an object 100, is presented as five embodiments of a lumbar tensioning orthosis 300, illustratively built upon the superior right planar base 117, superior left planar base 116, and inferior 110 planar bases used in the first thoracic embodiments. Many parts in the following lumbar embodiments are shaped, positioned, and used in a manner that allows for a modular set of pieces to be fabricated (the bases to general categories of body types), which may be used to treat either the lumbar, thoracic, or any specific part along the spine.

However, modularity is not specifically the realm of this specific teaching but the reuse of images and parts is immensely useful in the presentation of simpler and easier to digest images, descriptions, and pats list, given the massive amount of potential variation that can be done. Indeed, even just connecting the turnbuckles from the thoracic to different parts of resized bases can displace the specific area of tensioning.

Springs also can displace the center point of tensioning and a few embodiments, over the next few pages, will demonstrate a sufficient breadth of examples that may aid in individual modifications for preference. And whereas a myriad of additional springs uses can be, even in a large range of tensioning apparatus or inter-base connections, the authors'perception of obviousness of these other potential alternative uses of springs in the device are illuminated in the claims to retain a much less cluttered and succinct legend, illustrative section, and number of images.

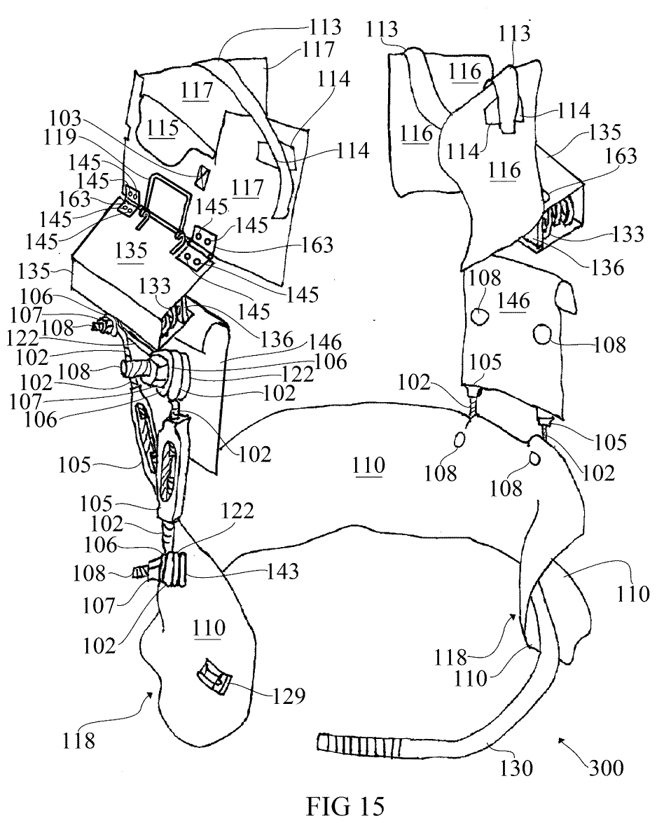

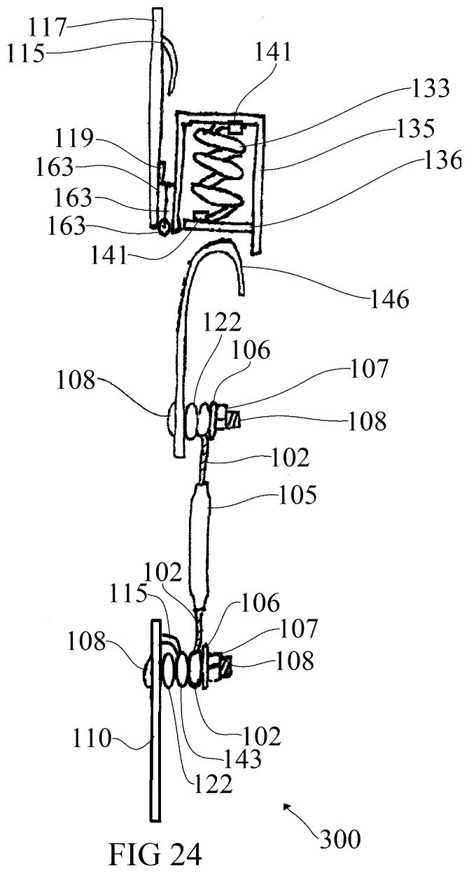

Thus, a fifth, sixth, seventh, eighth, and ninth variant/embodiment of the general embodiment of the general tensioning orthotic device 100, is pictured in FIG. 15-29; and these specifically address the need for tensioning of the lumber: a mobile, lumbar tensioning orthosis, however, adjustments to the center area of tensioning can be done on any part of the body where this device can be used. For simplicity and consistency of expression, and therefor ease of understanding, these variants will continue to be referred to as lumbar tensioning orthotics.

The claims, much more broad by nature, will provide other examples in a manner that allows for a capacious set of choices in how one would like to craft a tensioning orthotic based on the lessons and concepts taught and demonstrated is an exhaustive number of potential alternatives, which would (having displayed sufficient knowledge and competence) fall under author's perceptions of obviousness; however it is impractical and overly financially burdensome (if not fiscally impossible considering claims cost structure) to lay out the surfeit of varying spring combinations, and multi-planar structures with more than 2 base levels, and combinations of these two novel applications of pressure diversion.

### First Embodiment of a Lumbar Tensioning Orthosis 300

The first embodiment of the lumbar orthosis variant 300, is pictured in FIG. 15, 20-22, 24, and 28, and is similar to the thoracic embodiments 200 seen in FIG. 4-13; however, a third and fourth base level are added (each composed of two bases, one for each side of the body). between the superior and inferior basses. These inferior middle and superior middle bases are composed of two compression spring housing bases/units 135 (for the superior middle base level) and two inverted-“j” shaped, inferior middle bases 146 (for the inferior middle base level).

This embodiment includes, pictured, superior left planar base 116, superior right planar base 117, inferior planar base 110, two superior-middle bases (here on labeled as “compression spring housing bases” 135), two inverted-“j” shaped, inferior middle bases 146, carriage bolts 108, compression springs 133, washers 106, hex nuts 107, cylindrical cross-rods 122, large turn buckle end rods 102, large turn buckles 105, hook 113 and loop 114 fasteners, ratchet buckle 129, ladder strap 130, carriage bolt holes 103, hinges 163, flat rectangular bar 136, Chicago screw sleeve nut 144, Chicago scree mating bolt 145, handle fold 115, hip flange 118, double coil torsion spring 119, spacers 143, and brads 141.

In this embodiment, the two compression spring housing bases 135 are made by constructing a planar base similar to the superior left planar base 116 and superior right planar base 117 planar bases, only smaller, and with a rectangular opening, constructed thusly. It is bent with two folds on one end such that, if it were parallel to the general plane of either the superior left planar base 116 or superior right planar base 117, it would bend from the body and then bend inferiorly, forming a squared opening on one end. This opening houses compression springs 133 and, thus, is referred to as a compression spring housing base 135.

In this embodiment, one end of each inverted-“j” shaped, inferior middle base 146 is rounded, superiorly, away from the body touching side, for wedging into the compression spring housing base 135, in which, several compression springs 133 sit, fastened by brads 141 to the inside of the opening, opposite the mouth of the compression spring housing base 135.

One of the superior polar ends of each of these compression springs 133 is fastened by brads 141. The other polar end of a set of compression springs 133, per side, attaches to the superior flat edge of a flat rectangular bar 136; this flat rectangular bar 136 is shaped so that it goes deeper in the hole in which its superior compression springs 133 emanate, as these compression springs 133 compress. In this manner, each of the two superior openings (one for each superior spring housing base), has, on the inside of the opening immediately opposite the opening, a flat rectangular bar 136 (generally shaped to and smaller than the opening) attached by compression springs 133.

The constructed compression spring housing units 135 are each, of two (one for each side) positioned above the new inverted-“j” shaped, inferior middle bases 146, and each is positioned at or just beneath the respective superior planar bases (superior left planar base 116 and superior right planar base 117), depending whether the inverted-“j” shaped, inferior middle bases 146 are fully engaged with the superior middle bases (the compression spring housing bases 135).

Along the bottom of the superior bases (superior left planar base 116 and superior planar right base 117) and the most near/associated compression spring housing base 135, hinges 163 are attached—one compression spring housing base 135 per superior base (superior left base 116 or superior right planar base 117).

When the hinges are folded upon their other half, the squared opening of the compression spring housing base 135 faces down. A double coil torsion spring 119 is attached to the inferior areas of the superior planar bases (superior right base 117 and superior left base 116) and between the hinges 163. One each side, these hinges and double coil torsion springs joins the compression spring housing base 135 and the inferior of the superior left planar base 116, if on the left side (or inferior of the superior right planar base 117, if on the right side) together with the attached compression spring housing base 135.

This final construction would give an appearance such that the compression spring housing base 135 is squared off (bent and squared toward the inferior when engaged (when the double coil torsion spring is tightened)) while the rest of this compression spring housing base 135 overlaps its respective superior planar base (superior left planar base 116 or superior right planar base 117), pressing upon these bases at any point just beneath the arm pit abutting handle fold 115. Depending on which portion of the lower back is being targeted, and other factors, the amount to which the compression spring housing base 135 covers the non-body abutting plane of the superior bases (superior left planar base 116 and superior rights planar base 117) varies as the size of the compression spring housing bases 135 can vary.

In this embodiment, the inferior of the two inverted-“j” shaped, inferior middle planar bases 146, is essentially squared and flat with exceptions to mild modifications (accounting for optional body curvature). The superior of these two new inverted-“j” shaped, inferior middle planar bases 146, is rounded out, away from that tensioned, in at least a generally, inverted “j” shape. The roundness or quasi-fold back on itself is of a manner that allows it to slide into the space created in constructing the compression spring housing base 135, the opening that faces inferiorly when swung up, or when engaged.

In this embodiment, the compression spring housing base 135 is essentially similar to the inverted-“j” shaped, inferior middle base 146, however its position (relative to the rest of the device) depends on inferior to superior pressure exerted upon it and upon its distal planar side. As inferior to superior pressure is applied, through the twisting of the large turnbuckle 105, the two inverted-“j” shaped, inferior middle bases 146 move superiorly as the ultimate inferior planar base 110 provides resistance. This pushes both the superior middle bases (the compression spring housing bases) 135 flat against the outside of the superior planar bases (superior left planar base 116 and superior right planar base 117), on each side used, as the inverted-“j” shaped, inferior middle bases 146 engage them. The superior of the inverted-“j” shaped, inferior middle bases 146, enters the opening of the compression spring housing base 135, engaging the compression springs and engaging the double coil torsion spring 119, pushing the two ends of the hinges 163, against themselves.

That is to say that this action slowly changes the orientation of the compression spring housing unit 135 wherein the opening of the housing unit goes from facing the plane of the superior planar base (superior left planar base 116 or superior right planar base 117) to being parallel with them, as the inferior middle base additions (inverted-“j” shaped, inferior middle bases 146) engages and enters the compression spring housing base 135 opening.

In this embodiment the hinges are attached using sized Chicago screws. The Chicago screw mating bolts 145 of a sex bolt, in some figures, is the only half of the Chicago screws'two parts (Chicago screw mating bolt 145 and Chicago screw sleeve nut 144) that can be shown due to the angle of the image.

In this embodiment, hook 113 and loop 114 fasteners are used as shoulder straps. A ratchet fastener/buckle 129 and ladder strap 130 are used at the front opening of the inferior planar base 110, as opposed to a band or hook 113 and loop 114 fasteners used in earlier embodiments.

In this embodiment, as with the thoracic embodiments 200, a mild flange 118 is made upon the inferior, front opening, of the inferior planar base 110. Additionally, the larger turnbuckle 105, larger turnbuckle end rod 102, washer 106, hex nuts 107, carriage bolt 108 are assembled as they were in the first embodiment of the thoracic embodiments 200, except that the top of the large turnbuckle end rod 102 is connected to the inferior of the inverted-“j” shaped, inferior middle bases 146, at approximately the same lateral spot, relative to the body, at which it was attached to the inferior of the two superior planar bases (superior left planar base 116 and superior right planar base 117) in the first embodiment of the thoracic tensioning device 200.

In this embodiment, depicted in FIG. 15, the carriage bolt holes 103 are retained in the superior left planar base 116 and superior right planar base 117 superior planar bases, as if the superior planar bases (superior left planar base 116 and superior right planar base 117) were reused in a modular capacity.

One mild, additional difference, in this embodiment, is that spacers 143 are used where the bulk of this hardware attaches to the inferior planar base 110. The spacer 143, here, is sized so that a carriage bolt 108 may pass through it and so that its thickness makes up for lateral thickness differences created by the aforementioned modifications.

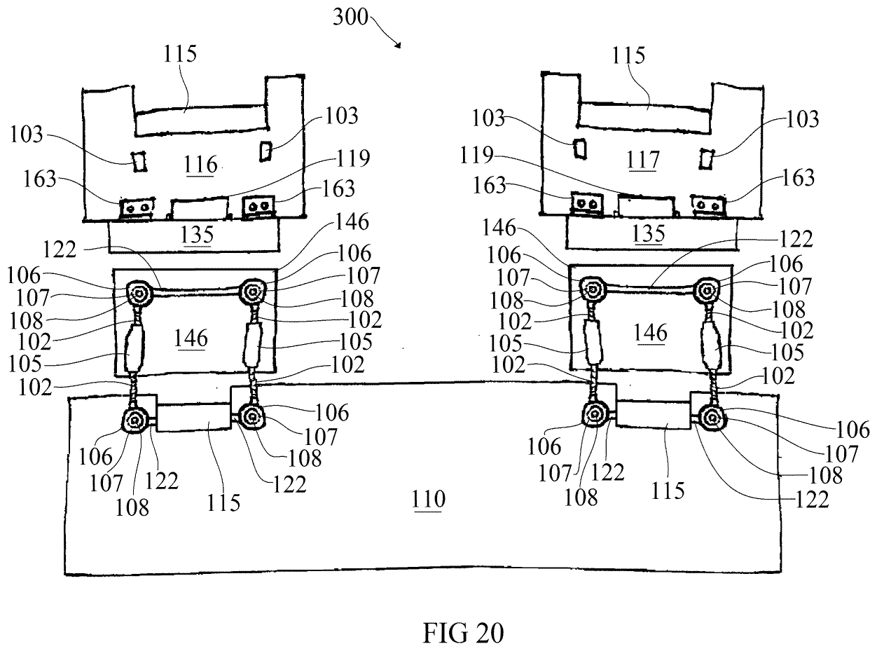

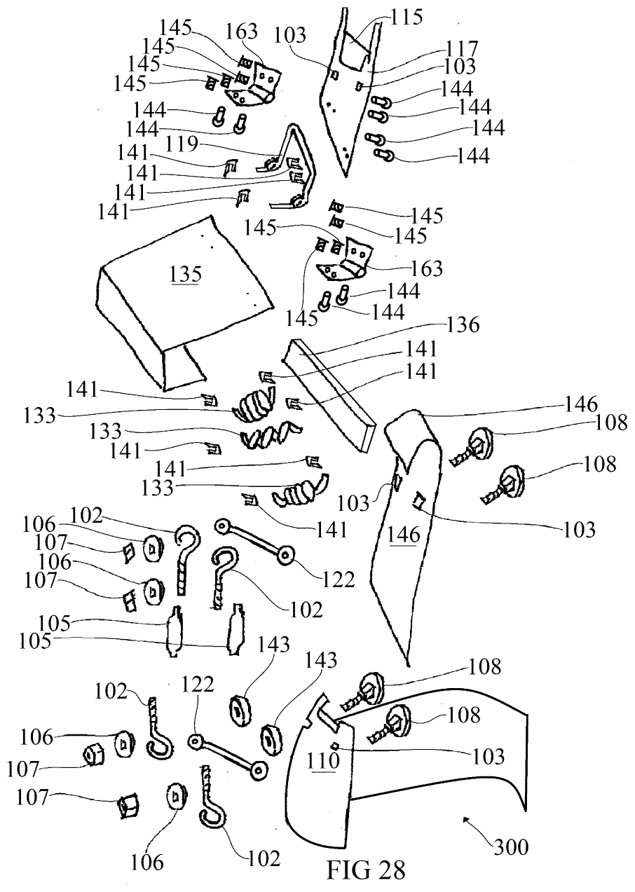

This embodiment is shown in various manners and from various angles; in FIG. 15 (a three dimensional front to back view), FIG. 20 (a flat view in line with the style of FIGS. 11), 21 and 22 (in line with the style of FIGS. 12 and 13), 24 (a profile view), a FIG. 28 (a blowout, collectively showing, clearly, how all the parts fit together). In some of these views, as with FIGS. 12 and 13, certain features are omitted in order to better display otherwise hidden angles of the most important features, or to not crowd the image with unnecessary additional labels.

For the remaining embodiments of the mobile lumbar tensioning orthosis variant 200, some of the additional angles of view are omitted, because they would provide no further insight than that already provided, due to the fact that variations predominately, between these embodiments, occurs in the middle section of the device, and do not need, for most of the alternates, an additional frontal, flat view.

Regarding clarity of that depicted the following is shared: the first embodiment of a lumbar tensioning 300 alternate of the general body tensioning orthosis 100, is seen in FIG. 15, wherein a three-dimensional frontal view is shown. In FIG. 20, a flat view is shown from outside the body and, because of the depiction of the washer 106, nothing beneath that is shown.

The blow-out, depicted in FIG. 28 shows all the omitted pieces, except the straps; and this figure (FIG. 28) shows how all pieces fit together, by showing all parts on the right side of the first embodiment of a lumbar tensioning orthotic, at and above the right (relative to user) side the inferior planar base 110.

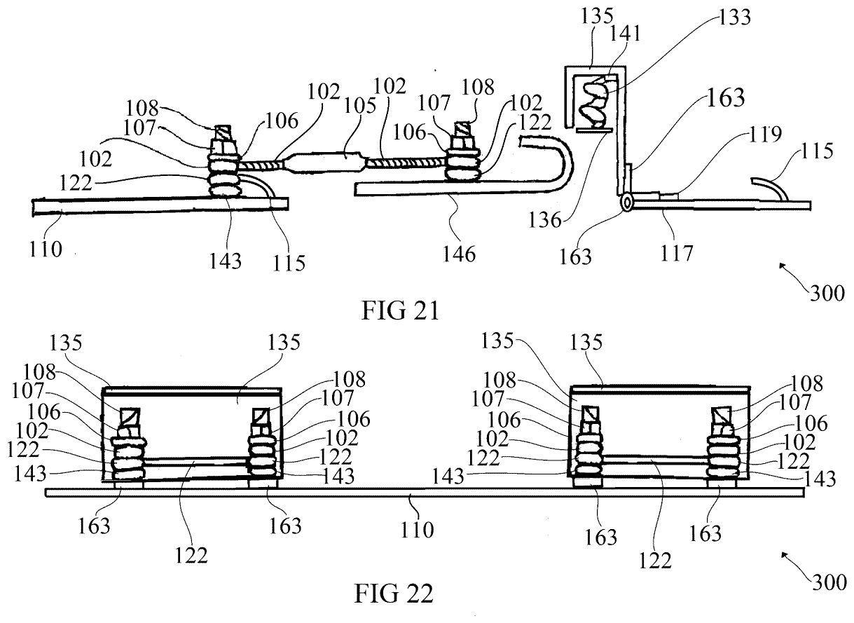

This first embodiment is further elucidated in FIG. 21 (a side profile representation of FIG. 20) and FIG. 22 (a bottom to top 90 degree rotation of FIG. 20). That is FIG. 21 displays a side profile of the flat view shown in FIG. 20, but, for clarity, the inverted-“j” shaped, inferior middle base 146 is shown as if it were properly curved (away from that being tensioned) for engaging the compression spring housing base 135, itself properly folded as would be, to demonstrate, from a new angle, how these two interacting pieces would interact.

Also, FIG. 22 displays a view of FIG. 20 along the plane of the planar bases, from the bottom to the top of the unit. This display omits curvatures of all bases, retaining the folds of the compression spring housing base 135. Additionally, the depiction omits the compression springs 133 and flat rectangular bars 136, for clarity in viewing the structure and for ease of understanding the art.

Due to the flat nature of the presentations in FIGS. 20, 21, 22, and 24, the pin-point pressure reducing flange 118, is only shown in FIG. 15.

### Second Embodiment of a Lumbar Tensioning Orthosis 300

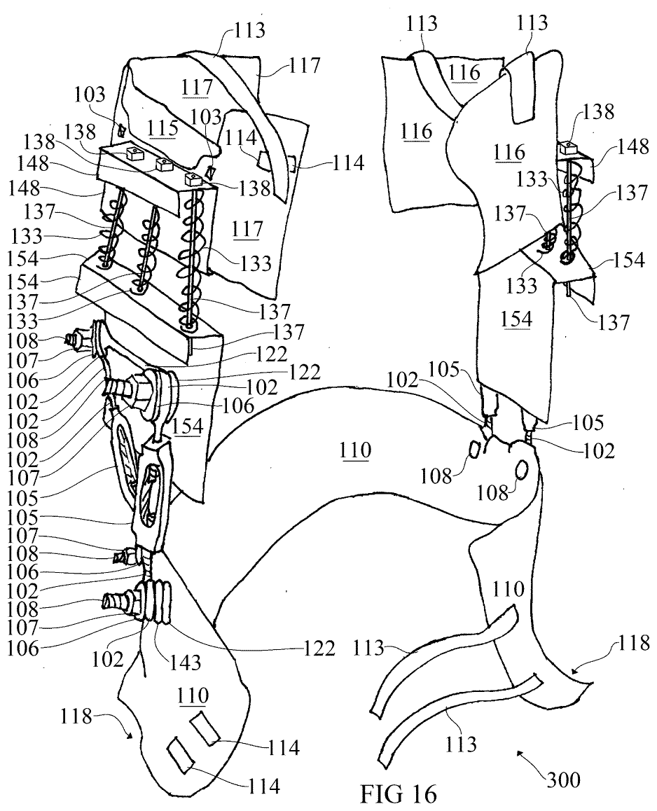

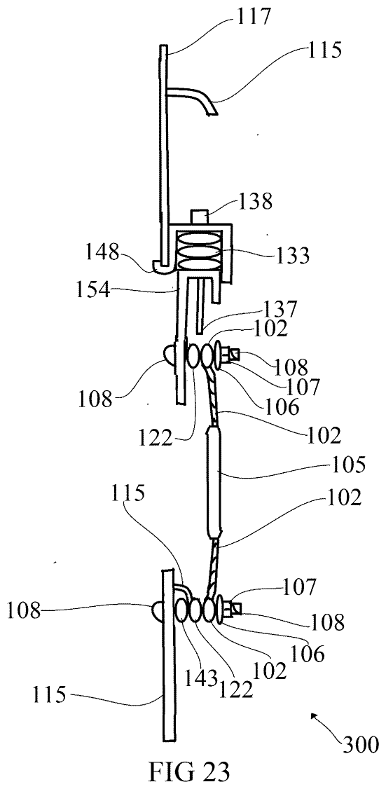

The second embodiment of the lumbar tensioning orthosis variant 300, of the general tensioning device 100, is shown in FIGS. 16 and 23, which show a three-dimensional, front view and a side, flat, profile, respectively. Constructing the embodiment is done by making modification to the first lumbar embodiment.

In this embodiment, the depictions include two “first alternate compression spring housing bases” 148, two generally cubed nuts with threading at their tops and bottoms (generally cubed nuts 138), compression spring 133, carriage bolt holes 103, guide pin rods 137, two first alternate, compression spring resisting, inferior middle bases 154 (which directly presses against the compression springs 133), spacers 143, flanges 118, carriage bolts 108, washers 106, hex nuts 107, large turnbuckles 105, large turnbuckle end rods 102, cylindrical cross-rods 122, and hook 113 and loop 114 fasteners, inferior planar base 110, and handle folds 115.

In this embodiment, the first alternate compression spring housing bases 148 (one for each side of the body) are constructed on one end as the modified special end was in the first embodiment of the superior middle/compression spring housing base 135, first depicted in FIG. 15. The portion that would have attached to hinges, now, instead, bends in the opposite direction as the squared folds and forms a small “u” shape, which is just big enough to slide onto the inferior edge of either superior planar base (superior left planar base 116 or superior right planar base 117), with the proximal end of the “u” shape fold of the first alternate compression spring housing base 148 lying on the proximal side of the superior planar base (superior left planar base 116 or superior right planar base 117) to which it attaches. This final construction/shape resembles a sort of asymmetrical “s” seen in the profile view FIG. 23 of the first alternate compression spring housing base 148.

If this “u” shaped fold is not tight enough itself to hold the relevant base against the superior left (or right, as the case may be), then the first alternate compression spring housing bases 148, may be affixed through one of several different standard methods.

In this embodiment, guide pin rods 137 are passed through the superior most plane of this first alternate compression spring housing base 148, and attached to a generally cubed nut 138 on the superior most portion of this plane. The generally cubed nuts 138 have threading on two opposing ends, so that these may be reused in other embodiments, for the truncation of the parts list and for added clarity of the teaching.

In this embodiment, the first alternate, compression spring resisting, inferior middle bases 154 are essentially identical to their immediately superior counterparts, first alternate compression spring housing bases 148, except that they may be a bit smaller, the body-longitudinal run may be longer and there is no need (nor is it depicted) of a “u” shaped fold on the inferior edge of the first alternate, compression spring resisting, inferior middle base 154.

Additionally, the first alternate, compression spring resisting, inferior middle bases 154 would have the guide pin rods 137 (previously attached above) passing through the superior plane of the top of the squared portion. That is to say, in this embodiment, the opposite ends of the guide pin rods 137 (not necessarily threaded) pass through a similar superior plane and part of the first alternate, compression spring resisting, inferior middle bases 154 and they go through the top flat portion of the inferiorly facing opening of the first alternate compression spring housing bases 148, where in the generally cubed nuts 138 fasten the longitudinally running guide pin rods 137 in place.

Through this structuring, the inferior polar ends of the guide pins move inferiorly along the distal planar side of the alternate inferior middle base (one on each side) as the first alternate, compression spring resisting, inferior middle bases 154 move superiorly, closer to the generally cubed nuts 138. The superior ends of the tensioning rod superior ends (large turnbuckle end rods 102 depicted) attach to the alternate inferior middle bases as they did to the inverted “j” shaped inferior middle base.

Between the axially planar distally projecting folds (which create the aforementioned openings) of each first alternate, compression spring resisting, inferior middle bases 154 and its superior most, corresponding first alternate compression spring housing base 148; the compression springs 133 are run along the guide pin rods 137, with the guide pins in the center of compression springs 133.

In this embodiment, the rest of this embodiment is assembled as, and essentially functions as the first embodiment for lumbar tensioning 300, wherein the twisting of the large turnbuckle 105 presses the superior and inferior planar bases against the protruding ends of the body being tensioned. The pressure upon the superior part of the body being tensioned is reduced compared to that endured by the inferior of the body being tensioned due to spring mechanics. This diverts the mid-point of tensioning inferiorly toward the lumbar.

Adjustments in the size of the parts and resistance of the springs can be made to further specifically target areas of the lower and even lower-mid back.

### Third Embodiment of a Lumbar Tensioning Orthosis 300

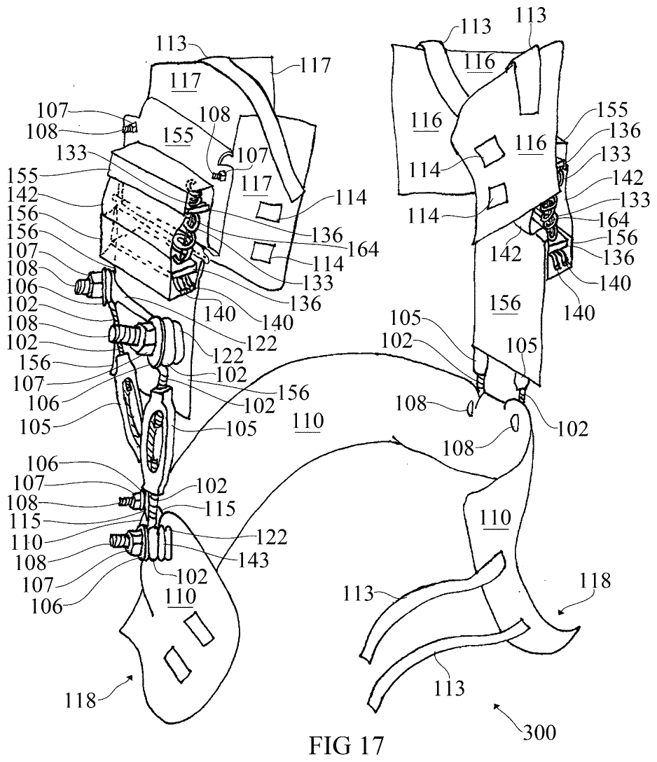

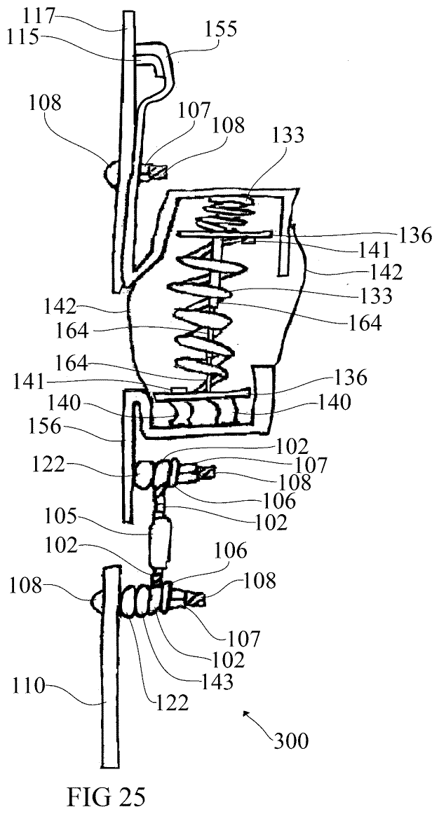

The third embodiment of the lumbar tensioning orthosis variant 300, of the general tensioning device 100, is shown in FIGS. 17 and 25 and shows a three-dimensional, front view and a side, flat, profile view, respectively. Constructing the embodiment is done by making modification to the second lumbar embodiment, with most prominent changes happening to the compression spring housing structures.

For this embodiment, seen in the depictions, there are two superior middle, second alternate compression spring housings 155, compression springs 133, telescopic guide pins 164, two second alternate, compression spring resisting, inferior middle bases 156 (alternates of inferior middle bases)—which houses the leaf springs 140—spacers 143, flanges 118, carriage bolts 108, washers 106, hex nuts 107, large turnbuckles 105, large turnbuckle end rods 102, cylindrical cross-rods 122, brads 141 and hook 113 and loop 114 fasteners, inferior planar base 110, and handle folds 115.

In this embodiment, the second alternate compression spring housings 155 are shaped like distorted s's with a one-sided, rounded serif on the bottom end of the “s”. That is to say, starting with a flat plane, the superior of this plane would curve towards that being tensioned, so as to lie over the handle folds 115 of the respective superior planar base (superior left planar base 116 or superior right planar base 117). The outside edges of this superior edge are cut out. So, that the entirety of the superior is not covered. This area would be the “serif” of descriptive metaphor used.

To continue constructing this base (the second alternate compression spring housing 155), for each base, moving away from the “serif”, near the middle, the base folds back on itself, away from that being tensioned. Further along this direction, it eventually does a 90 degree fold away, again away from that being tensioned, and then, finally, does another fold 90 degrees inferiorly. These last folds form an inferiorly facing squared opening into which springs will be housed as described below—similar to the earlier compression spring housing bases'135 attachments to flat rectangular bars 136 through the use of compression springs 133, in the first embodiment of a lumbar tensioning orthoses 300.

This is how the second alternate compression spring housings 155 are depicted. Obvious alternative means of construction can be more simply executed; but, for the general continuity of the general methods used, for clarity, this method of attaching to the previously used bolt holes 103 of the superior planar bases is done to demonstrated potential modularity of the teachings.

In this embodiment, each, of the two, second alternate compression spring housings 155 are affixed to the superior right planar base 117 and superior left planar base 116 superior bases, with carriage bolts 108, in the manner and location that the top of the large turnbuckle rods 105 were previously affixed to these superior planar bases (superior left planar base 116 and superior right planar base 117) in the first embodiment of the thoracic embodiments 200, reusing the carriage bolt holes 103.

In this embodiment, the second alternate, compression spring resisting, inferior middle bases 156, starting with a generally rectangular planar base, at its superior most position, folds back upon itself (away from that being tensioned) and then folds superiorly twice, forming an inverted spring housing section similar to the spring housing section of the second alternate compression spring housings 155, described above. To re-iterate, this now squared inferior opening (one on each side per second alternate, compression spring resisting, inferior middle base 156) is superior-to-inferiorly inverted, so that the opening of the second alternate, compression spring resisting, inferior middle bases 156, face up towards the second alternate compression spring housing 155.

In this embodiment, the distal and proximal—as positioned against a tension-intended torso—portions of the openings (of these two new couples of alternate bases (one couple with a inferiorly facing openings, and the other with the opposite)) are connected by a pliable, flat, rectangular material (possibly a cloth) 142, to keep the compression springs 133 from pinching the user of the device.

In this embodiment, inside each squared opening of the second alternative compression spring housing 155 and the second alternate, compression spring resisting, inferior middle base 156, there is a flat rectangular bar 136. On each of two superior flat rectangular bars 136, one for each side of the device, there are compression springs 133 attached to the flat rectangular bars 136. The opposite end of each set of compression springs 133 are attached, one on each side, to the second alternate compression spring housing 155 specifically on the inside of the opening, directly opposite that opening.

On each side, the inferior of the superior rectangular flat bar 136 (on each side) attaches to the relevant and nearby, superior side of an immediately inferior flat rectangular bar 136. This is done through the use of compression springs 133 and telescopic guide pins 164, which go through the compression springs 133 between the two flat rectangular bars 136, on each side of the body being tensioned.

The inferior of each inferior couple of flat rectangular bars 136 abuts against a set of leaf springs 140. Each set (two sets total) of leaf springs 140 attaches to the left or right second alternate, compression spring resisting, inferior middle base 156, one set per side. The attachment of each set of these leaf springs 140 is made against the area opposite the mouth of the squared opening of the second alternate, compression spring resisting, inferior middle base 156.

To restate the position of inferior flat rectangular bar 136 and springs attachments, for clarity; on each side of the body the inferior flat rectangular bar 136 is connected to compression springs 133 on the superior side of these two flat rectangular bars 136. The opposite side of these two flat rectangular bars 136 abut against the tops of leaf springs 140 on their inferior side. The leaf springs 140 are attached to each of the two (one per side) second alternate, compression spring resisting, inferior middle bases 156, on the inside of the inferior most portion of each second alternate, compression spring resisting, inferior middle base's 156 squared opening.

The superior rectangular flat bars 136, one per side, attach, by way of compression springs 133 to the inside of the superior most portion of the squared opening of the second alternate compression spring housing 155. The top and bottom flat rectangular bars 136 (one couple on each side) attach to each other by means of compression springs 133 and telescopic guide pins 164.

The compression spring connection fasteners are pictured as brads 141, in FIG. 25; although alternative means of connection are easily done.

In this embodiment, the large turnbuckles 105 still attach, as in previous embodiments, to the inferior base 110. That is to say, in this embodiment, the main tensioning hardware attaches as normal to the inferior base 110; however the upper, large turnbuckle end rods 102, which are opposite this inferior base 110 connection (along with the normal washer 106, hex nut 107, carriage bolt 108 and cylindrical cross-rods 122) attach to the second alternate, compression spring resisting, inferior middle bases 156.

In this embodiment, hook 113 and loop 114 fastener straps are shown, and they act as they do in aforementioned embodiments, as shoulder straps holding and to hold close the opening in the inferior base 110.

In this embodiment, spacers 143 are used as they were in the last embodiment.

In this embodiment, use of the embodiment is pretty straight forward now, based upon previous embodiments, and simply requires the use of superior-inferior pressure, in the depicted figures, provided by twisting the large turnbuckles 105, while the device 300 is worn.

Twisting of the large turnbuckles 105 presses the superior and inferior planar bases against the protruding ends of the body being tensioned. The pressure upon the superior part of the body being tensioned is reduced compared to that endured by the inferior of the body being tensioned due to spring mechanics. This diverts the mid-point of tensioning inferiorly toward the lumbar.

Adjustments in the size of the parts and resistance of the springs can be made to further specifically target vertically divergent areas of the lower back.

### Fourth Embodiment of a Lumbar Tensioning Orthosis 300

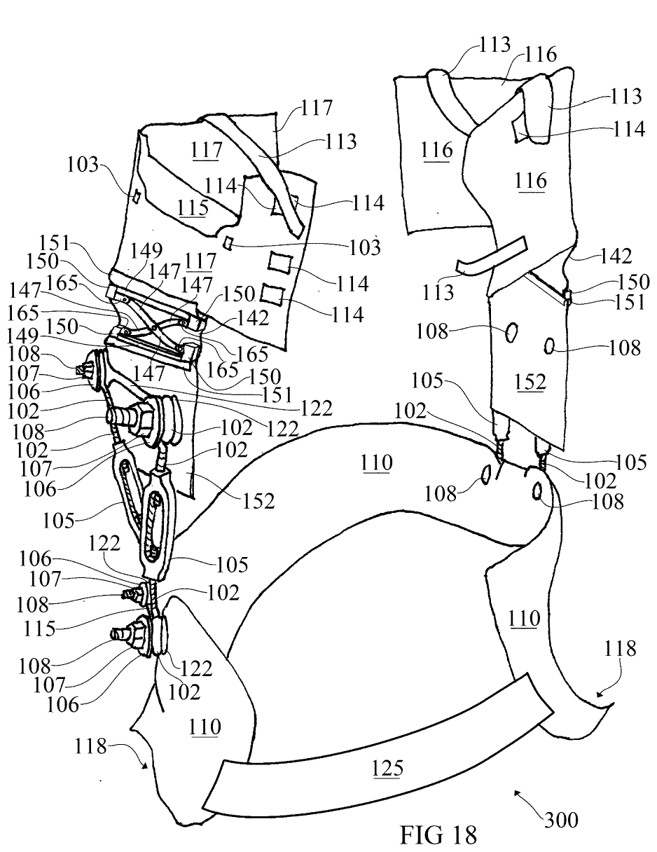

A forth embodiment of the lumbar tensioning orthosis variant 300 (a second variant of the general tensioning device 100), is shown in FIGS. 18, 19, and 26, and shows a three-dimensional, front view; a blow-out of the spring structure; and a side, flat, profile, respectively.

In this embodiment, the objects shown are compression springs 133; two flat middle bases/“flat, unfolded intermediary base” 152; flanges 118; carriage bolts 108; washers 106; hex nuts 107; large turnbuckles 105; large turnbuckle end rods 102; superior planar bases (superior left planar base 116 and superior right planar base 117), carriage bolt holes 103 (due to illustrative re-use of these), cylindrical cross-rods 122; and hook 113 and loop 114 fasteners, an inferior planar base 110; an elastic band 125, a pliable, flat, rectangular material (possibly a cloth) 142; and handle folds 115, rods with a channel through which a nodule may slide (“channel rod” 149); end caps for said channel rods (caps for channel rods 150); hotdog bun/“U” shaped bases 151 (upon which attaches the channel rods 149) cylindrical rods with a protruding nodule 165, “hole[s] for rivets or screws/sex bolts” 166, rivet fasteners 134, spacers 143, Chicago screw sleeve nuts 144, and Chicago screw mating bolts 145, crossing cylindrical rods (“scissor rods”) 147, with three holes each for rivets or screws/sex bolts 166 (for creating a single scissor joint and four swivel joints), per two crossing scissor rods 147.

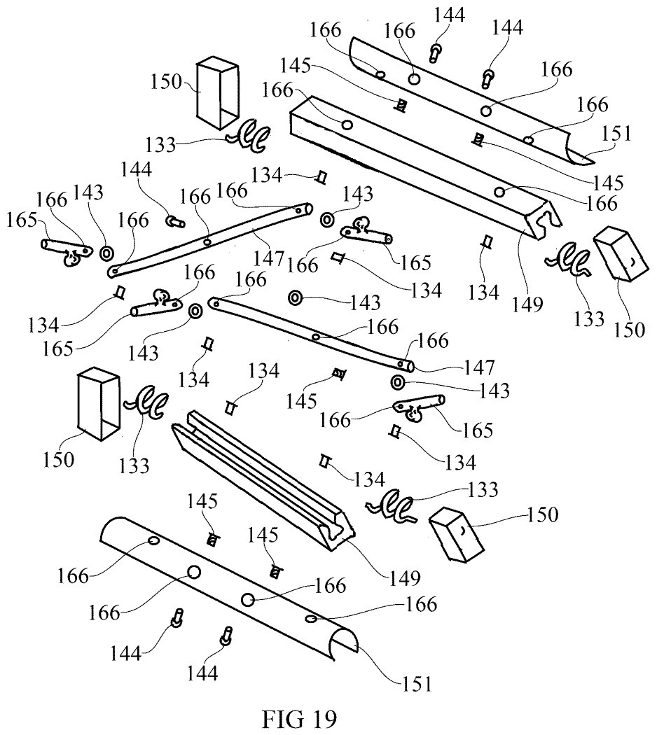

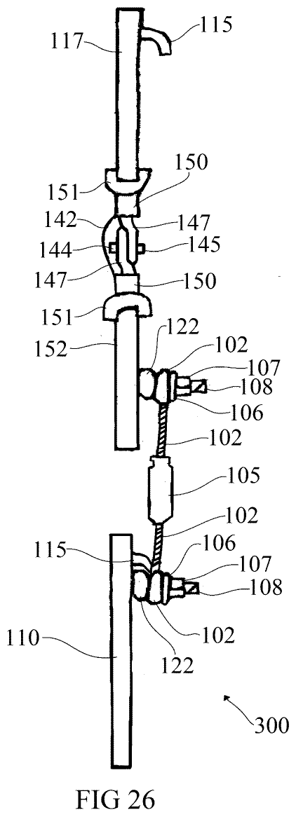

This embodiment is depicted by FIG. 18, a three-dimensional view of the front; a flat profile view from the side, FIG. 26; and FIG. 19, a blow-out of the mechanism unique to this embodiment. Constructing the embodiment is done by making modification to the third lumbar embodiment, removing all that is not specified in the previous paragraph, and the compression springs (to be reintroduced shortly in different spots.

To construct this embodiment, one slides the open side of a “u” shaped base 151, onto the inferior edges of both superior planar bases (superior left planar base 116 and superior right planar base 117). The other side, the closed side, of the “u” shaped bases 151, attaches, to a channel rod 149 through the inside of the “u” and through the interior of the channel (the channel opening facing the opposite direction of the connected “u” opening). These two channels house nodules of a cylindrical rod with a protruding nodule 165, one on each side of each channel. (In the final construction, these nodules (two per channel) each, when the device is actuated, slide the length of half of the channel, from a resting position in the center its corresponding channel rod 149.)

One end of these cylindrical rods with a protruding nodule 165, the side without the protruding nodule, has a hole for rivets or screws/sex bolts 166 in it, which attaches to each of four crossing cylindrical rod with three holes, id. est (“i.e.”), scissor rods 147. These four attachments per couple of crossing scissor rods 147, are made with rivet fasteners 134 and spacers 143 in order to make four swivel joints per side of the body/per superior planar base (superior left planar base 116 or superior right planar base 117).

Therefore, there should now be four swivel joint (connecting the end of a scissor rod 147 to one, non-noduled, end of a cylindrical rod with a protruding nodule 165) connections per each of the two scissor joints or per two crossing scissor rods 147.

If each “x of the crossing scissor rods 147, were laid against each side of the body the openings of the “x” would lie so that each opening faces either up, down, posteriorly or anteriorly.

At the two superior ends and two inferior ends of the “x”, there would be a connection, anterior and posterior, to each cylindrical rod with a protruding nodule 165.

The channel rods 149 would be positioned so that one channel rod 149 is at the top and bottom of each “x”, so that, on each side (inferior or superior), per each of two crossing scissor rods 147, the two inferior nodules would slide into the relevant inferior channel and two superior nodules would slide into their respective superior channel.

That means, constructed, there are four superior-most cylindrical rods with a protruding nodule 165: two per superior channel rod 149 (one superior channel rod 149 per couple of crossing scissor rods 147 per superior planar base (superior left planar base 116 or superior right base 117)). The same is true for four inferior most cylindrical rods with a protruding nodule 165.

Put another way, inside each channel rod 149 slides the nodule of two of these cylindrical rods with a protruding nodule's nodules 165. Each channel rod 149 is attached to a “u” shaped base 151, of which there is one channel rod for the inferior edge of the superior left planar base 116 and one channel rod 149 for the inferior edge of the superior right planar base 117, attached by means of these “u” shaped bases 151. Similarly, there is one channel rod 149 for the superior edge of a flat, unfolded intermediary base 152, one on the left side and one on the right. Each of these two flat, unfolded intermediary base 152 sit under a couple of crossing scissor rods 147, one per side of the body being tensioned.

Working from top to bottom, on each side, the order of parts would be the superior planar base (superior left planar base 116 or superior right planar base 117), a “u shaped base 151, a channel rod 149, a couple of cylindrical rods with a protruding nodule 165 (sliding in the channel rod 149), crossing scissor rods 147, another couple of cylindrical rods with a protruding nodule 165, a channel rod 149, a “u shaped base 151, a flat, unfolded intermediary base 152. The flat, unfolded intermediary base 152 is then attached to the inferior planar base 110 through tensioning rods (a large turnbuckle 105 attached to two large turnbuckle end rods 102.)

(Note the new middle bases (one for each side) are the only bases on a level between the most superior and most inferior level of middle bases.)

Each lateral side of the channel rods 149 have a cap (cap for channel rod 150), which prevents the nodule from sliding out, and which houses a compression spring 133, against which the cylindrical rod with a protruding nodule 165, opposite the swivel joint, press, sliding into the center of the compression springs 133, which abut against a nodule.

(Note at this juncture that “noduled side” does not necessarily preclude this side from externally housing a compression spring, as it does (the rod passing through the center of compression spring 133 to which it abuts, on the side opposite the swivel joint, the compression spring abutting against the nodule.)

Two couples of scissor rods 147, overlap and connect to each other at their respective middles, through holes for rivets or screws/sex bolts 166, by means of a Chicago screw mating bolt 145 and Chicago screw sleeve nut 144, sandwiching a spacer 143, which creates a scissor joint on each side of the device 300, at the two “scissor joints” mentioned above (mid-way along the length of each of two crossing scissor rods 147).

Put another way, in this embodiment, rivet fasteners 134 fasten the “u” shaped bases 151 to the channel rods 149. The outer ends of the scissor rods 147 are attached to cylindrical rods with a protruding nodule 165 as previously outlined, themselves (cylindrical rod with a protruding nodule 165) attached, as pairs, to each channel rods 149. The channel rods 149 each attaching to a unique “u” shaped base 151, by means of rivet fasteners 134.

In this embodiment, the two inferior “u” shaped bases, are each attached to the superior end of one of two new middle bases (called “flat, unfolded intermediary bases”) 152, each positioned directly inferior to the superior planar bases (superior left planar base 116 and superior right planar base 117), one per superior planar base.

As such, working from the top to bottom, in this embodiment, each side of the superior planar bases (superior left planar base 116 and superior right planar base 117) have a “u” shaped base 151 attached to them. These bases each attach to a channel rod 149, which attach to cylindrical rods with a protruding nodule 165, which attach to crossing scissor rods 147 which attach to more cylindrical rods with a protruding nodule 165, which attach (by sliding in the channel) to channel rods 149, which attach to a lower inverted “u” shaped base 151 which then, finally attaches to a flat, unfolded intermediary base 152 (again, on each side) which then attaches by tensioning hardware (like the large turnbuckle 105 and large turnbuckle end rod 102) to the ultimate inferior planar base 110. At each large turnbuckle end rod 102 connection a cross-rods 122 connects to another laterally proximal large turnbuckle end rods 102 (one per each middle base, per side of the inferior planar base 110), a large turnbuckle 105 connecting each couple of large turnbuckle end rods 102, inferiorly to superiorly.

In this embodiment, the superior portion of each middle base (flat unfolded intermediary base 152) has a pliable, flat, rectangular material (possibly a cloth) 142, attached to the “u” shaped base's 151 skin abutting side. One pliable, flat, rectangular material (possibly a cloth) 142 per side per two “u” shaped bases 151. This prevents the joints from pinching the user of the device 300.

In this embodiment, although hook 113 and loop 114 straps are shown, they act as they do in past embodiments, except upon the inferior planar base 110 opening.

As has been standard in these embodiments, a pressure diverting flanges 118 at the inferior front of the inferior planar base 110 are depicted in the three-dimensional view FIG. 18; and they are at the front closure of the inferior planar base held drawn closed, post donning, with an elastic band 125.

To clarify, in this embodiment, the main tensioning hardware attaches in the manner it has been shown to do in the other embodiments, to the inferior planar base 110; however the superior end of the large turnbuckle 105 and large turnbuckles end rod 102, combo is attached, along with the normal washer 106, hex nut 107, carriage bolt 108 and cylindrical cross-rods 122, to a flat unfolded intermediary base 152.

In this embodiment, use of the embodiment is pretty straight forward now, based upon previous embodiments, and simply requires the use of properly spaced superior-inferior pressure, in the depicted figures, provided by twisting the large turnbuckles 105, while the device 300 is worn.

Twisting of the large turnbuckle 105 presses the superior and inferior planar bases against the protruding ends of the body being tensioned. The pressure upon the superior part of the body being tensioned is reduced compared to that endured by the inferior of the body being tensioned due to spring mechanics. This diverts the mid-point of tensioning inferiorly toward the lumbar.

Adjustments in the size of the parts and resistance of the springs can be made to further specifically target areas.

### Fifth Embodiment of a Lumbar Tensioning Orthosis 300

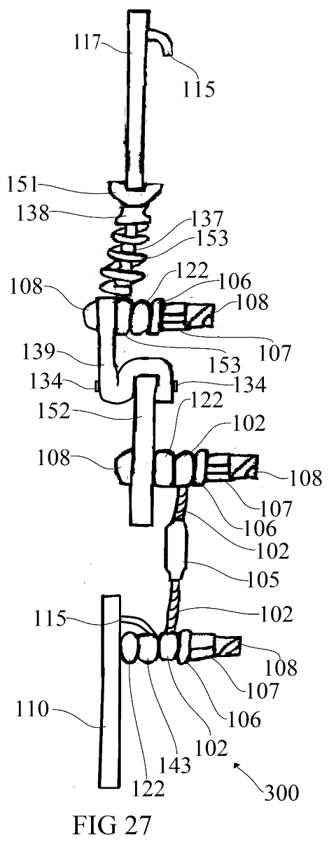

The fifth embodiment of the lumbar tensioning orthosis variant 300, of the general tensioning device 100, is shown in FIG. 27 showing a side, flat, profile of the right side. This embodiment is also shown in FIG. 29, which shows a blow-out of the other side, for simplicity of depiction—both sides are equivalent. This includes the depicted side's connection to the inferior planar base 110.

The depictions, of one side of the symmetrical device (above the inferior planar base 110), includes an inferior middle base (a flat, unfolded intermediary middle base 152); a flat planar superior middle base, with its inferior edge folded into a “u” shape 139; a “u” shaped base 151; two generally cubed bolts with threading at their tops and bottoms (generally cubed nuts 138). There are also two modified compression springs, which increase in thickness superiorly to the inferiorly (i.e., compression spring rods 153).

These compression spring rods 153 have, axially/transversely (to user of device) planar holes, at their tops for guide pin rod 137 insertion. The compression spring rods 153 also have sagittal (to the perspective of the user of the device) planar holes at their bottoms for carriage bolts 108.

Finally, there are guide pin rods 137; spacers 143; carriage bolts 108; washers 106; hex nuts 107; large turnbuckles 105, large turnbuckle end rods 102; carriage bolt hole 103, rivet fasteners 134; a pliable, flat, rectangular material (possibly a cloth) 142; small flat-headed fasteners 162; cylindrical cross-rods 122; inferior planar base 110, handle folds 115; superior planar bases (i.e., superior left planar base 116 and superior right planar base 117); and holes for rivets or screws/sex bolts 166.

For simplicity of demonstration, hook 113 and loop 114 fasteners are not depicted, but they would act as they have in past embodiments.

In this embodiment, working from top to bottom, one starts with a superior planar base (either the superior left planar base 116 or the superior planar right base 117) on either side of the body being tensioned. These are akin to the previous ones used in all previous embodiments besides that of the general tensioning orthotic 100. Again, there are two nigh-identical sides, a superior left planar base 116 and a superior right planar base 117.

In this embodiment, one side of this variant's construction will be stated, starting at the superior left (to the user's perspective) planar base 116 and working down to the left of the inferior base 110, the rest of which is depicted to show the entirety of the inferior planar base 110 part, for clarity.

In this embodiment, the other half of this embodiment, will therefore, simply be the same but working from the right superior planar base 117 to the inferior planar base 110. The depiction (in FIGS. 29 and 27) also illustrate this methodology of presentation of the teaching at hand.

In this embodiment, the superior left planar base 116, as constructed in past embodiments, with carriage bolt holes 103, and handle folds 115, is attached to a “u” shaped base 151, which runs a portion of the anterior to posterior length of the inferior edge of the superior left planar base 116.

In this embodiment, the opening of the “u” shaped base 151 houses the inferior most portion of the superior left planar base 116.

In this embodiment, the inferior most asymptote, of this this “u” shaped base 151, attaches to two generally cubed nuts 138. This connection is made with hardware through this inferior facing asymptote.

In this embodiment, this is done by using small flat-head fasteners 162 on the inside of the “u”. These face towards the bottom and go through the most inferior asymptote of the “u” shaped base 151. On the other side of this pass through, the small flat-head fasteners 162 attach to cubed shaped bolts (generally cubed nuts 138), two in this depiction.

In this embodiment, the opposite end of the cubed shaped bolts, which are not attached to the small flat-head fasteners 162, is attached, through threaded holes, to guide pin rods 137, threaded at one end.

In this embodiment, these two compression spring rods 153 increase in thickness as they follow the guide pin rod 137 in the inferior direction. At the bottom, the spring juts out to a side and forms an “o” shape through which a carriage bolts 108 passes. This “o” shape is flat in a generally sagittal manner (flush against the exterior/distal surface of a flat planar superior middle base, with its inferior edge folded into a “u” shape 139).

The top of the spring is axially planar. So, the guide pin rod 137, may pass through most of the spring, passing the inferior “o” shape as the device is actuated. This jutting out of the compression spring rods 153 (one, for the two on one side, posteriorly and the other, on that side, anteriorly) to the side allows for the compression spring rods 153 to compress without the guide pins being blocked. That is to say that the inferior longitudinal ends of the guide pin rods 137 are able to move close to its side's flat, unfolded intermediary base 152 and longitudinal pressure from the application of the tensioning rods, which pushes this flat, unfolded intermediary base 152 superiorly, away from the inferior planar base 110.

To re-iterate, at this point, in this embodiment, the inferior “o” of the two compression spring rods 153 (on the side discussed) abut against a “a flat planar superior middle base, with its inferior edge folded into a “u” shape” 139. Upon the side of the inferior, sagittal planar “o's”, of the inferior of the compression spring rods 153, (the side which does not abut against the “a flat planar superior middle base, with its inferior edge folded into a “u” shape” 139 end) there sits a cross-rod 122, connecting the inferior ends of the two compression spring rods 153 to each other. At this point there are also washers 106 and hex nuts 107 attached to the carriage bolts 108, which pass through a flat planar superior middle base, with its inferior edge folded into a “u” shape 139.

(Note the superior cylindrical cross-rod 122, one on each side, may be between the inferior fold of a flat planar superior middle base, with its inferior edge folded into a “u” shape 139 and the compression spring rod 153 OR between the washers 106 and the compression spring rods 153. In the later position, blockage of the guide pin rods'137 movement may be avoided by simple alterations to the manner in which the inferior ends of the compression spring rods 153 jut out.)

In this embodiment, the inferior “u” shape, of a flat planar superior middle base, with its inferior edge folded into a “u” shape 139, has the superior edge of a flat unfolded intermediary base 152 (inferior middle base), inserted into its opening. These two middle bases (on each side) are affixed to one another with rivet fasteners 134 through holes for rivets or screws/sex bolts 166.

In this embodiment, a pliable, flat, rectangular material (possibly a cloth) 142, connects the a flat planar superior middle base, with its inferior edge folded into a “u” shape 139, to the superior most “u” shaped base 151, on the side of each which would be closest to the body being tensioned, preventing the springs and mechanisms from pinching a user of the device.

In this embodiment, large turnbuckle end rods 102 attach to laterally opposing sides of the unfolded intermediary base 152 at a point beneath the superior edge of the flat, unfolded intermediary base 152.

In this embodiment, as has been standard, a cylindrical cross-rod 122 connects the two large turnbuckle end rods 102 used upon this planar base (and similarly at the inferior large turnbuckle end rod 102 connections at the inferior planar base 110. Through the carriage bolt holes 103 of the flat, unfolded intermediary planar base 152 and the inferior planar base, at the side of the flat, unfolded intermediary base 152, passes carriage bolts 108, affixed with hex nuts 107 and washers 106.

In this embodiment, on the inferior end of the superior large turnbuckles end rods 102 is attached large turnbuckles 105. On the inferior of these turnbuckle rods is attached a lower large turnbuckle end rods 102, which attach to another lower cylindrical cross-rod 122, which runs between the holes of the two large turnbuckle end rods 102 on this side of the embodiment, of the inferior planar base 110. At this point, at this base level, there are also spacers 143 and the standard carriage bolt, hex nut 107 and washer 106 connection to the left side of the inferior planar base.

In this embodiment, all this is mirrored on the right side using the superior right planar base 117, which ultimately connects to the right side of the inferior planar base 110. A profile view, depicting this right side, is shown in FIG. 27.

In this embodiment, use of the embodiment is pretty straight forward now, based upon previous embodiments, and simply requires the use of properly spaced superior-inferior pressure, in the depicted figures, provided by twisting the large turnbuckles 105, while the device 300 is worn.

Twisting of the large turnbuckle 105 presses the superior and inferior planar bases against the protruding ends of the body being tensioned. The pressure upon the superior part of the body being tensioned is reduced compared to that endured by the inferior of the body being tensioned due to spring mechanics. This diverts the mid-point of tensioning inferiorly toward the lumbar.

Adjustments in the size of the parts and resistance of the springs can be made to further specifically target areas.

### Appendage Tensioning Embodiment 400

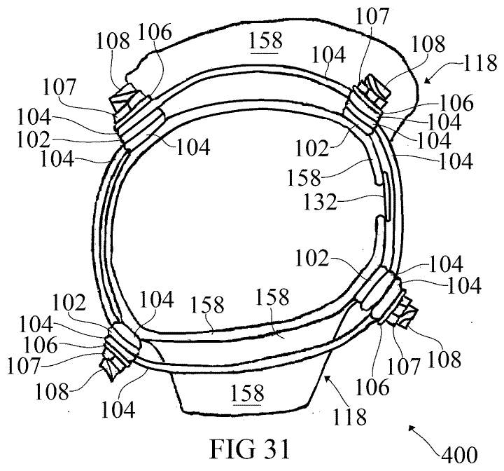

A fourth variant of the general embodiment 100, is an orthosis which tensions parts the appendages 400, and is specifically displayed in FIGS. 30 and 31,

For ease of comprehension, the lower of two legs are shown depicted in FIG. 30. The device is the same for each leg. FIG. 31 shows top down axial plane view of one of this device. This embodiment will be described as if only describing one of the two displayed sides.

In the embodiment the depiction includes a proximal base wrap for appendage 157; a distal base wrap for appendage 158; flanges 118; carriage bolts 108; washers 106; hex nuts 107; large turnbuckles 105; large turnbuckle end rods 102; curved, rounded, curved cross-rods 104 (rounded to a quarter of the circumference of the tensioned body/object to be tensioned 101), stitch-closure buckles 131, stitch-closure strap 132, and the tensioned body part/object to be tensioned 101.

This embodiment is constructed exactly as the general embodiment 100 except that two targeted body parts/object[s] to be tensioned 101 are shown (the left and right leg), the top of the mechanism (left or right variant) comprises the proximal base wrap for appendage 157, and its contrary is the distal base wrap for appendage 158. Both wraps have a single disunion which are held together by stitch-closure buckles 131 and stitch-closure straps 132, and a flange 118 is made at the top of the foot, the device molded to the shape.

Quarter of circumference lying curved, rounded cross-rods 104 are also used at each large turnbuckle end rods 102 connection to the planar bases, using carriage bolts 108, washers, and hex nuts 107. Nut variants are not used.

FIG. 30 shows a 3-d view of the device and FIG. 31 shows a view through the center of the wraps'circles (proximal to distal) without the object to be tensioned 101 being present.

As such, in this embodiment, tensioning happens by twisting the large turnbuckles 105.

### Cervical Tensioning Embodiment 500

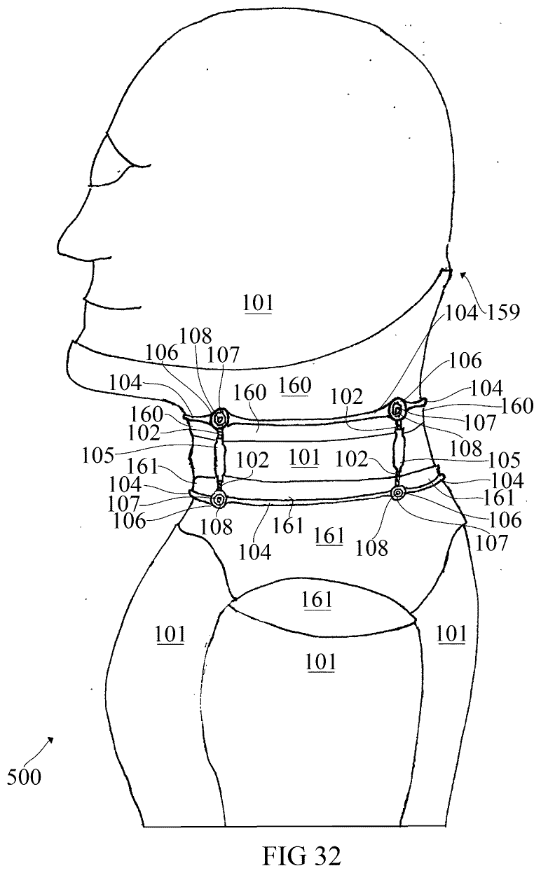

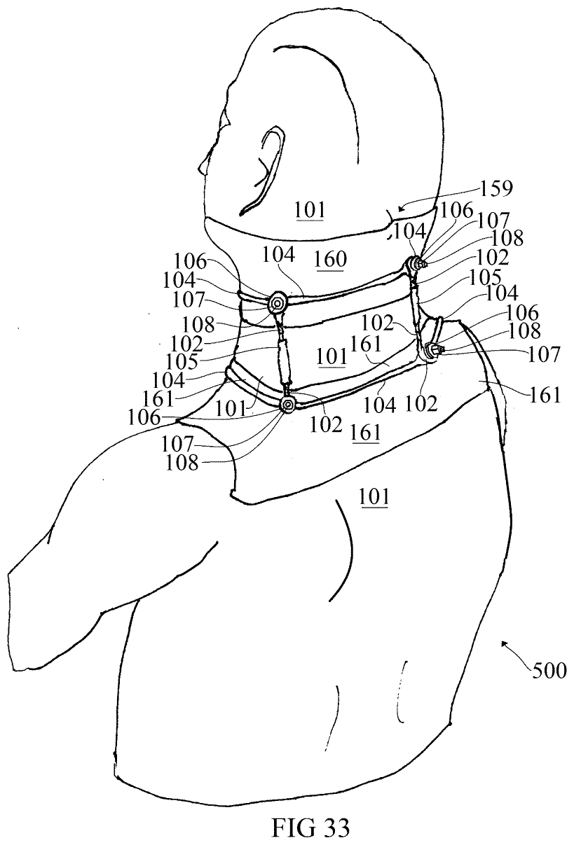

A fifth variant of the general embodiment 100, tensions the cervical spine, specifically displayed in FIG. 32 and FIG. 33, showing an embodiment of a device for tensioning the cervical spine 500 with a full 360 degree axial application.

The depictions includes superior cervical base mold 160 around the jaw and occipital bun 159, inferior cervical base wrap 161, large turnbuckles 105, large turnbuckle end rods 102, washers 106, hex nuts 107, carriage bolts 108, 1/4 of the body circumference curved, rounded cross-rods 104, occipital bulb 159, and tensioned body part/object to be tensioned 101.

This embodiment is identical to the general embodiment 100 except that the superior wrap is a superior cervical base mold 160 and the inferior most base is a shoulder conforming wrap/support base (inferior cervical base mold 161). For simplicity, each planar support base is a single, unbroken molded wrap around the body.

As is with the general embodiment 100, all large turnbuckles 105, large turnbuckle end rods 102, curved, rounded cross-rods 104 washers 106, hex nuts 107, and carriage bolts 108 are used.

This cervical device uses more than two linear pressure inducing apparatuses for the full neck tensioning, and it uses rods which laterally connect the tops and bottoms of these linear tensioning apparatuses. This collectively distributes pressure along the circumference.

Nuts may be placed within the turnbuckle assemblies to retain desired distance longer than otherwise.

Additions of other aspects of this may be optionally applied including diversion of center point of tension pressure methods.

### Combination Tensioning Embodiment 600

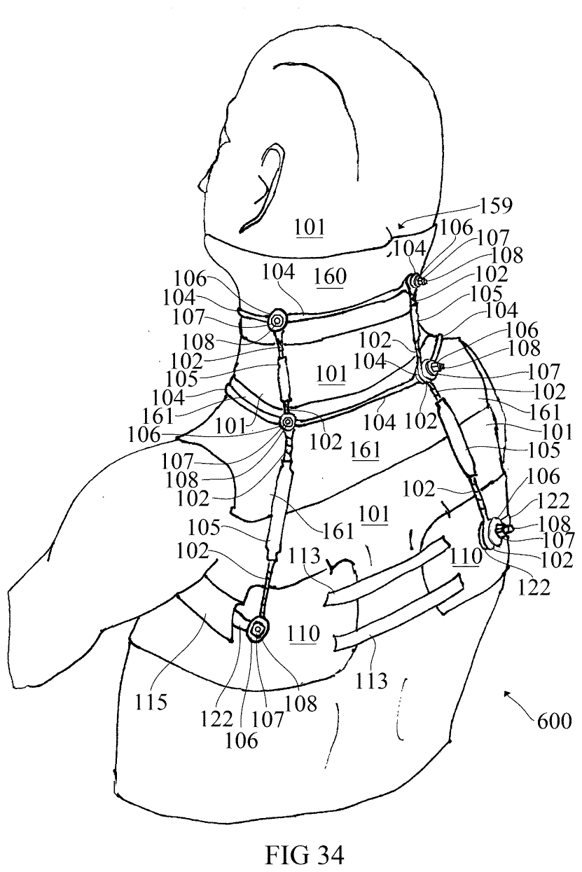

A sixth variant of the general embodiment 100, tensions the cervical spine, and upper back simultaneously and is displayed in FIG. 34, which shows an embodiment of a device for tensioning two areas simultaneously 600.

The device is exactly the same as the cervical spine tensioning embodiment 500, but, beneath this embodiment, beneath the arm pits, is attached an inferior planar base 110 as presented above, which specifically has an opening (herein at the back of the targeted body/ object to be tensioned 101) connected by hook fasteners 113, and handle folds 115.

Although the opening is inverted, modularity comes into play because this full wrap with one opening, COULD be a superior wrap in earlier embodiments, replacing the left and right variants shown above, and fitted to different groupings of body shaped at this point.

This embodiment shows how multiple parts can be tensioned and also how a slight modifications can be made to press against other lesser protrusions

A modification would be to have the generally body conforming base sit beneath the breast, of the corpulent, slightly contrary to that depicted in FIG. 34, and do any manner of previous embodiment re-alignments, in order to use the tops or bottoms of corpulent pecs/breast as additional pressure resisting surfaces, without muscle atrophying body constrictions, in line with the general embodiment of this teaching 100, oblique or lateral cross bars may be used along any number of bases levels to further modulate tensioning pressure to the desired point, as shown elsewhere.

In this embodiment 600, the reused inferior planar base 110 is attached to the shoulder conforming wrap/support base (inferior cervical base mold 161) by means of large turnbuckles 105, large turnbuckles end rods 102, washers 106, hex nuts 107, and carriage bolt 108. Additionally, rounded cylindrical cross-rod 122 connects large turn buckles end rods 102, at the front and rear of each arm pit, and pass under handle folds 115.

Additionally, shown in FIG. 34, depicting this embodiment, are quarter circumference lying cross-rods 104, the occipital bun 159, and a superior cervical base mold 160, around the jaw and occipital bun 159.

As is with most every other embodiment, tensioning is provided by twisting the large turnbuckles 105.

All shared through descriptions and generalities to this point has been enough to sufficiently, using the teachings above, to tension the entirety of the spine down to the lower lumbar, without a lack of mobility and without constriction of the torso or neck, in addition to the tips of the fingers and toes, diverting the center of tensioning pressure through many different means.

Although some spring mechanism and multi-base linear pressure inducing apparatus shifts have been shown, which divert this center tensioning pressure, many different constructs and combinations can be devised, far too many for one to illustrate even half. Indeed, there is a massive array in alternative tensioning inducement mechanism available, broadly referenced in the claims.

As such the concepts displayed in the several different illustrated concepts, and some further examples in the claims, should sufficiently guide one in the construction of exceedingly specific devices to target whatever specific part of the body they desire, excluding the sacrum and skull, without anchoring to bones and without a loss of blood flow, while the user remains mobile for a long time.

Although the device has been explained in relation to various simply illustrated embodiments, it is to be understood that many other possible modifications and variations can be made without departing from the spirit and scope of the invention, as will be seen in the claims.

The best has been done here to demonstrate the general concepts and advanced applications upon many different areas of use. In this embodiment, these specifications might aid in the recreation of the teaching and to aid in its construct and understanding uniquely necessary applications of the teachings to craft embodiments to target very specific areas in a personalized use to a mass manufactured modular embodiment.

1. An indirectly attached, mobile (regarding the movement of a user—to which the device is indirectly attached—as being free from a fixed location) worn device useful in the tensioning of a body to be tensioned, without atrophy from prolonged use, without needing (ceterus parabus) surgical intervention to use the device, without constricting blood flow to a debilitating level, and without the need of weights (i.e., historically used traction systems), comprising;
(a.) a light-weight, rigid, panel planar base on an axial plane of the body to be tensioned (i.e., a “base level”), which laterally lies (partially or totally) over the circumference of a body to be tensioned; wherein, regarding a minimum core structure of two diametrically opposed planar bases for a minimum functioning of the device,
(i.) there is a minimum of two diametrically opposed planar bases such that at least the longitudinal ends (ends which are longitudinally furthest from the center of an area to be tensioned) of the minimum, requisite two planar bases are on their own distinct base level (i.e., for the minimum structural requisite of two planar bases, one end of one of the planar bases on the distal side of a body to be tensioned and one end of the other of the planar bases on the proximal side of a body to be tensioned; or one end of one of the planar bases on the inferior side of a body to be tensioned and one end of the other of the planar bases on the superior side of a body to be tensioned--terminology dependent on the location being tensioned, and the location of the other opposing planar base),
(ii.) a minimum of two diametrically opposed planar bases are, when the device is used, simultaneously put under pressure to move apart, which pushes parts of a body to be tensioned apart through the contact of the planar bases with the body to be tensioned;
(b.) connecting two or more planar bases, a linear-pressure inducing apparatus or linear-pressure inducing apparatus assembly (manual, semi-automated or automated), driven by any force, exempli gratia (e.g. non-exhaustive list of examples);
(i.) Screw-based linear actuator assembly, e.g.,
(A.) a ball screw assembly,
(B.) a worm gear based assembly (e.g., most simplistically rods attached by pivot joints to the pinion of a simple worm gear actuator, wherein the rods extend from the center of the pinion when the pinon is rotated 90 degrees);
(ii.) tooth and groove linear actuator assembly, e.g.,
(A.) a rack and pinion based apparatus used with a crank and a stopper (e.g., spring loaded plungers, tie-down strap, ratchet and pawl, and indexing pins),
(B.) a ratchet and pawl based apparatus coupled with a pinion and rack apparatus;
(iii.) telescoping linear actuator assembly e.g.,
(A.) chain driven linear actuator,
(B.) Segmented Spindle actuator;
(iv.) cam and crank assembly (e.g., a modified Cr émone bolt with an indexed stopper);
(c.) a pressure ameliorating structure such as a pressure ameliorating change to a planar base's shape, change in a planar base's conformity to the body, a pressure ameliorating attachment, a combination of these or other such structures, e.g.;
(i.) planar base curvatures conforming to the contours of a body to be tensioned, over which the modified planar base lies,
(ii.) planar base large curvatures or folds (optionally spreading out from point of conformity) at some of the edges furthest from the center of that being tensioned and/or areas where the planar bases would push into large protrusions of a body to be tensioned or a prosthetic,
(A.) Wherein the curves or folds roll or fold away from the body to be tensioned, e.g., sharply change direction, suddenly round-off in a roll, or taper off in a roll (flange out) (potentially also extending along these areas enough to divert pin-point pressure to the degree necessary as increasing pressure is applied),
(iii.) gel padding,
(iv.) moving the points of contact of a pressure inducing apparatus and/or adding extensions onto a planar base away from the base level (to shift the center of tensioning pressure),
(v.) moldings the planar bases to body to be tensioned contours away from the site of tensioning pressure's most extreme engagements with the body to be tensioned,
(vi.) toggled pressure ameliorating base adaptations attached (directly or indirectly) to non-tensioning target-zone areas of a body to be tensioned,
(vii.) a body-to-be-tensioned-protrusion conforming and pressure diverting structure attached to a flat planar base one of its ends,
(viii.) a planar base composite structured like a hammock, wherein an elastic band connects the longer edges of two rectangular planar bases, distributing pressure received over a large area (e.g., used at an arm pit, wherein the mesh is inside an arm pit and the planar bases are at the superior and anterior of the shoulder, lying over the arm and torso).

2. A device according to claim 1, wherein said linear pressure inducing apparatus are spaced, in the case of more than one being used, along the circumference of a general center of the part of a body to be tensioned; and are a manually driven (by muscles) linear tensioning rod assembly; e.g.;
(a.) turnbuckle assembly with varying end fittings, e.g.,
(i.) clevis
(ii.) eye,
(iii.) oblique,
(vi.) swivel rod;
(b.) tension rods with springs in their structure, e.g.;
(i.) simple telescopic tensioning rod assembly that twists to unlock, elongates, and then twists to lock, housing compression springs internally;
(ii.) complicated tensioning rod assembly using a worm gear and hand crank,
(A.) which drive a screw rod, which pushes a nut along the screw,
(B.) which pushes a telescoped sheeting rod, which attaches to a cylindrical bolt,
(C.) which has a rod telescopically housed in its opposite side,
(D.) which passes through a saddle clasp attached to a second object, wherein a compression spring wraps the last rod between the saddle clasp and the telescopic opening,
(E.) wherein the nut is held in place by a laterally displaced telescoping rod attached to the nut and primary planar base or base of the worm gear;
(iii.) complicated tension rod assembly consisting of a modified worm gear-driven screw rod, featuring a crank knob directly attached to oblique helical gear (the tip of which traces a spiraling groove in the screw,
(A.) wherein the screw rod advances (for the primary extension phase) in length as the knob turns, wherein a thicker rod is hard attached to the end of the screw rod, where in this thicker rod has a smaller telescopically housed bar in the end opposite the screw rod,
(B.) wherein this smaller telescopically housed bar is a flexible round rack attached to a pinon (the pinion to be attached to a second object),
(C.) wherein, during the second extension phase (the pressure displacement phase) a tension spring is attached to the thicker rod housing the housed bar and the middle of the pinion,
(D.) wherein the rack pulls the housed bar (rack) from its telescopic housing, wherein the entire mechanism is stabilized by a stopper plug plunger equipped with a spiral spring to provide calibrated mechanical resistance at the rack-and-pinion interface.

3. A device according to claim 1, further comprising, for the lateral terminal ends, when there is a break to the totality of a planar base's lay over a circumference, (or between other ends of planar bases) a plurality of linking hardware options, e.g.,
(a.) a hook and loop fastener(s),
(b.) a stitch buckle and stitch fastener(s),
(c.) an elastic band,
(d.) no hardware/no connection.

4. A device according to claim 1, further comprising one or more springs (of uniform or varying progressions of thickness) which either, as a collection springs (predominately as a singular linear pressure inducing assembly), induce pressure upon the bases; or, individually or with other springs, mitigate area induced pressure or divert the center of tensioning upon a body to be tensioned; e.g., tension springs (i.e. extension springs); e.g., one or more tension springs (symmetrically or asymmetrically used along the circumference of a body to be tensioned (e.g., a tension spring mediating the connection of one end of said linear pressure-inducing apparatus and any planar base)), torsion springs (e.g., used in a manner that pressure induced by another means stores the pressure locally through the torsion springs such that total device center of tensioning pressure is diverted), compression springs (e.g., one or more compression spring(s) (symmetrically (longitudinally or laterally) or asymmetrically (longitudinally or laterally) used around a body to be tensioned, directly or indirectly upon a planar base, directly or indirectly mediating pressure exchanges between two or more planar bases), spiral springs (e.g., spiral spring used with a crank a stopper and a wheel, which joins two rods (attached off center from the middle of the wheel) that are linearly extendable by rotating the wheel), conical springs (e.g., conical springs attached on one end to a longitudinally extreme end of a base with its other end under a protrusion-conforming (pressure-distributing) medium); exempli gratiīs (e.gg¹);
(a.) springs used within (symmetrically (laterally or longitudinally) or asymmetrically (laterally or longitudinally) the structure of a linear pressure inducing apparatus, e.g., a modified turnbuckle assembly that uses a modified turnbuckle with a helical threaded end, spiral springs, indexing stopper and round ratchet with helical spiraled axel, a notched rod with stopper, a rectangular bar with a rectangular opening on one half of its plane, a saddle clasp and an extension spring,
(i.) wherein a female ending of a modified turnbuckle attaches to an eye end rod,
(ii.) wherein the other end of the modified turnbuckle is a male rod with helical threading,
(ii.) wherein the helical threading of the male rod end of the modified turnbuckle lies next to a helical threaded axel, which attaches to a center of ratchet wheel (with an stopper) attached to a spiral spring (so that the spring is tightened as the ratchet turns),
(iii.) wherein, opposite the axel, as a second axel, attaches a long rod, the bottom of which attaches to (one side) a lateral flat rectangular bar, the other lateral side of which has a rectangular opening,
(iv.) wherein a longitudinal end of a long rod with notches (a notched rod) slides through the rectangular bar and the other longitudinal end of the notched rod passes through a saddle clasp,
(v.) wherein an indexing pin stopper attaches to the clasp and follows notches on one side of the notched rod (preventing the rod from reversing as the notched rod passes through the saddle clasp,
(vi.) wherein the opposite end and side of the notched rod is similarly notched with the inside of the square opening engaging one of the notches, driving the notched rod away from the modifier turnbuckle as the turnbuckle is engaged,
(vii.) wherein the bottom of the notched rod attaches to an extension springs, which attaches to the saddle clasp,
(viii.) wherein the round ratchet stopper is released to re-loosen the modified turnbuckle while the rectangular rod repositions itself at a higher notch on the notched rod;
(b.) springs used as the predominate portion of a linear pressure inducing apparatus, e.g., compression springs, around telescopic guide rods, are placed around the circumference of a body to be tensioned (between two planar bases),
(i.) wherein the longitudinal ends of the springs and guide rods connect the planar bases,
(ii.) wherein ratchet buckles and ladder straps attach near the ends of the telescopic guide rods and pull the two bases towards each other,
(iii.) wherein the straps are released after the device is donned;
(c.) springs used to mediate connections between two bases, e.g., beginning with a superior planar base that partially lies over a torso's circumference at the area under one arm pit (laterally centered upon the arm pit) and an inferior planar base (which lies around the full circumference of an area that includes the lower abdomen and hips), at the space between these two bases, the bases are joined together by spring-loaded draw latches attached to both bases,
(i.) wherein the now inferior planar base holds a single cylinder pneumatic linear actuator and a battery power source,
(ii.) wherein this linear actuator connects to the superior planar base with a rod;
(d.) springs used as a composite of mechanisms attached to a planar base (the composite mechanism mediating pressure induced by another base (directly) or through another composite mechanism (indirectly)), e.g., beginning with a superior planar base that partially lies over a torso's circumference at the area under one arm pit (laterally centered upon the arm pit), and an inferior planar base, which lies around the full circumference of an area that includes the lower abdomen and hips, and upon the inferior planar base is a rod with notches that run up one of its sides,
(i.) wherein this rod is held to the inferior planar base with saddle clasp and crank on a gear (with indexing pawl and stopper forming a combo of a rack and pinion and ratchet and pawl),
(ii.) wherein upon some portion of the rod, above the clasp, there is a radial protrusion (which runs 360 degrees of the circumference of the rod), which resists a spring above this protrusion,
(ii.) wherein the other end of this spring runs into a flat square surface into which the rest of the rod telescopes,
(iv.) wherein there are four other planes attached to this square surface and all these planes form a rectangular prisms to which extension springs are attached, which run to and attach to the superior planar base (the inferior edge of which is just beneath this prism),
(v.) wherein this prism telescopes into a larger rectangular prism and the top square of the smaller prism (which inserts into the larger prism) compresses a spring inside the larger prism,
(vi.) wherein the larger prism is attached to the superior planar base by means of brackets that allow the device to move up and down the side of the superior planar base,
(vii.) wherein a portion of the planar base under the armpit does a curved, rounded fold (a roll) over just above this connection and goes over a lateral running cross-rod (which presses the inside of the roll when the device is engaged),
(viii.) wherein this cross bar is attached on each of its ends to an inferior to superior running channel rod (which attach to the superior planar base) and the top of the large prism (protrusion or not) presses against this cross-bar,
(ix.) wherein the posterior and anterior end of the cross-rod have superior moving rods that pass the shoulder and which are wound by compression springs,
(x.) wherein the superior of these superior moving rods attach to lateral rods, which attach to a planar base mold that wraps and conforms to the neck and jaw line,
(xi.) whereby pressure from the crank induces the large prism up the side, which pushes against the springs of the superior moving rods, which eventually leads to the large prism movement pushing the cross-rod into the dorsal side of the superior planar bases'roll;
(e.) springs used as a composite of mechanisms in conjunction with a new planar base on a new base level, acting in totality as a linear pressure inducing apparatus, e.g., beginning with an inferior planar base lying around the hips and a superior base partially lying over the circumference just beneath one arm pit, and another base partially lying over the same circumference as the superior base but upon a lower base level (a middle base); there is a single cylinder hydraulic actuator attached to the superior base with a fluid reservoir slung over the opposite shoulder,
(i.) wherein a rod indirectly attaches the actuator to the middle base,
(ii.) wherein this connection is done with a 90 degree elbow upon a cube,
(iii.) wherein the cube is inside of a rectangular prism that has a channel (through which the rod attaches to the cube) running down one of its side,
(iv.) wherein the cube is held up by a spring inside the prism,
(v.) wherein the bottom of the middle base attaches (via a rod) to a larger rod through a telescopic opening (the inserted smaller rod wrapped by a spring),
(vi.) wherein an inferior telescoping rod attaches to the other end of the larger rod,
(vii.) wherein the inferior telescoping rod attaches to the inferior planar base through an a saddle clasp with a closed bottom,
(viii.) wherein this final rod is wrapped in compression spring just above the clasp.

5. A device according to claim 1, further comprising more than one said planar base on a base level, e.g.,
(a.) a left and right superior planar base for each armpit, with gaps between them on the anterior and posterior of the torso,
(b.) planar base molds on opposite sides of large contours or protrusions of a body to be tensioned in order to induce resistance in two directions (simultaneously tensioning multiple areas of a body to be tensioned),
(c.) a small 6 inch by 6 inch planar square attached at the posterior and anterior of the right superior planar base pit to obliquely extend the longitudinal and lateral range of a planar base.

6. A device according to claim 1 further comprising a cross-bar that connects two or more linear pressure inducing apparatuses.

7. A device according to claim 1, wherein, alternatively, there are two or more linear-pressure inducing apparatus assemblies per sagittal or lateral splice of that being tensioned, e.g., upon the torso the left side of a body has two and/or more and the right side has two or more; OR the anterior has two or more and/or the posterior has two or more.

8. A device according to claim 2, further comprising more than one said planar base on a base level, e.g.;
(a.) specifically worn around the torso, the device comprises a single inferior planar base (which lies over the circumference of the lower belly and hips and which has a single disunion at the front, held together by straps), two non-overlapping superior planar bases (a left superior planar base and a right superior planar base) on the same base level (each laterally centered under the left or right armpit), and four of a turnbuckle assembly as the linear tensioning rod assembly;
(i.) wherein the superior planar bases have their lateral terminal ends held together upon their superior terminal ends by shoulder straps;
(ii.) wherein the turnbuckle assemblies are body-to-be-tensioned length (longitudinally) run (superior planar base to inferior planar base);
(iii.) wherein the turnbuckle assemblies are positioned in pairs per side of the body to be tensioned;
(iv.) wherein one pair of turnbuckles assemblies attach to the left superior planar base and another pair attaches to the right superior planar base and the other half of the turn buckles attach to the inferior planar base just beneath these connections;
(v.) wherein, of each pair of turnbuckle assemblies, a superior end on one is upon a para-coronal plane anterior the shoulder and the other is upon a para-coronal plane posterior the shoulder (i.e. on each side, one is positioned anterior the lateral plane marking the termination of the shoulder and one is similarly positioned posteriorly to the shoulder) and the superior longitudinal ends of the turnbuckle assemblies are attached where they lie to its side's respective superior planar base;
(vi.) wherein between these anterior and posterior attachments upon each superior edge of the superior planar bases, the bases are cut and this cut (one for each superior planar base) is molded to the conformity of the armpit in a curved fold or roll over, distally away from the body to be tensioned and this mold is padded with pressure reducing like mediums;
(vii.) wherein the inferior planar base, in the front of the torso, upon its lower edge between the disunion, is a slowly tapering away roll from the body (a flange) (a mild roll to account for superior movement of the thighs as one sits while wearing the device);
(viii.) wherein the inferior and superior end of each couple of turnbuckle assemblies is attached to the their complement of their pair by an optional cross bar (at least two cross bars for the inferior planar base and at least one cross bar per superior planar base and one or more of the longitudinal extremes of any planar base has a portion of their structure folding over a cross-bar;
(b.) As described in 8(a.); however, alternatively, the superior ends of the posterior couple of turnbuckle assemblies switch the superior middle base to which they are attached, per the anterior or posterior side upon which they are attached and the superior ends of the turnbuckle assemblies are held together so the superior planar base stays in position when one raises their arms above their head, e.g., by means of latch-able and rotating cross bars;
(c.) As described in 8(a.), however, alternatively, there are no cross bars and tensioning is intended just for one sagittal half and the right side turnbuckle assemblies and the right side superior planar base is removed,
(i.) wherein, additionally, a tensioning springs mediates the connection of the inferior ends of the turnbuckle assemblies'connections to the inferior planar base, lying over the distal plane of the inferior planar base.

9. A device according to claim 4, further comprising more than one said planar base on a base level.

10. A device according to claim 4, further comprising a cross-bar that connects two or more linear pressure inducing apparatuses.

11. A device according to claim 5, further comprising one planar bases modified such that this planar base is composed of alternating (laterally, longitudinally, or inter-weaving) strips of rigid and pliable (possibly elastic mesh) materials used to displace pressure upon pressure points or help displace center of tensioning pressure.

12. A device according to claim 6, further comprising one planar base modified such that this planar base is composed of alternating (laterally, longitudinally, or inter-weaving) strips of rigid and pliable (possibly elastic mesh) materials used to displace pressure upon pressure points or help displace center of tensioning pressure.

13. A device according to claim 8, further comprising one or more springs (of uniform or varying progressions of thickness) which either, as a collection springs (predominately as a singular linear pressure inducing assembly), induce pressure upon the bases; or, individually or with other springs, mitigate area induced pressure or divert the center of tensioning upon a body to be tensioned and a cross-bar that connects two or more linear pressure inducing apparatuses; e.g.;
(a.) As described in 8(a.), however, alternatively, the cross bar is not optional, there are two couples of planar bases between said superior planar bases and the inferior planar base (one of each couple for the left side of the body to be tensioned and one of each couple for the right side of the body to be tensioned and these new bases are rectangular with the longest side running parallel to the inferior-to-superior run of the torso; one of the couples per side (between the inferior planar base and relevant side's superior planar base) positioned in a manner that one of these two new bases, on each side, is superior to the other on that side of the body being tensioned (e.g., there becomes two superior middle bases and two inferior middle bases, in the manner that there are two superior planar bases, one middle base (superior of inferior middle) for each side of the body to be tensioned) and the superior middle bases change to become a couple of spring housing mechanisms and the inferior middle bases change to properly engage the springs when pressure is induced upon them;
(i.) wherein the four middle bases fold in a manner that there is an inferiorly facing opening at the top of each base (the opening on distal plane of the base, the side that does not touch the body to be tensioned), which is created by:
(A.) having the superior end of each middle base (inferior-middle bases and superior-middle bases) folded so that this fold is made perpendicularly to the plane of the torso and juts out, away from the body to be tensioned,
(B.) making, just past this fold (moving in the direction away from the fold, away from the body to be tensioned ), another fold, which makes another externally (in the same direction as the former fold) lateral 90-degree rotation—leaving an inferiorly facing gap, at the superior end of each middle base,
(C.) ensuring there is a slight difference between the superior middle base folds and inferior middle base folds, which is that the inferior middle base folds may slide into their respective (per the side of the body to be tensioned on which they exist) superior middle base folds;
(D.) attaching a series of compression springs in each opening of the superior portions of the superior middle bases (the length of the compression springs matching the orientation of a torso and their superior ends attached to the surface inside the squared opening, specifically the fold which is axially parallel to the body to be tensioned, opposite the openings'mouths (a spring housing opening));
(E.) attaching the inferior ends of the compression springs to a flat, rectangular bar, generally the length and width of the spring housing opening, in which the springs are seated, one flat, rectangular bar per side of the body to be tensioned, per superior opening;
(F.) ensuring the superior fold of each inferior middle base is positioned in a manner in which it would press against the immediately superior rectangular bar and, indirectly, the immediately superior compression springs (on its side of the body to be tensioned) if pushed superiorly;
(ii.) wherein for each of the two inferior middle bases, the superior end of the turnbuckle assemblies attach beneath their former attachments to the superior planar bases with a cross rod per two turnbuckles per inferior middle base;
iii.) wherein the inferior of the turnbuckle assemblies attach to the inferior planar base as prior;
(iii.) wherein the left side's superior-middle base (i.e., left spring housing mechanism) attaches to the left superior planar base AND the right side's superior-middle base (i.e., right spring housing mechanism) attaches to the right superior planar base such that:
(A.) attaching each superior-middle base (i.e., spring housing mechanism) to (centered) the most inferior edge and the distal plane of its nearest, most superior planar base by using hinges and a double coil torsion springs,
(B.) making the other half of the connection (upon the superior middle base (i.e., spring housing mechanism)) upon the plane of the superior middle base (i.e., spring housing mechanism) which is opposite that of the folded opening (also done upon the longitudinally contrary end of the plane that is opposite the opening),
(C.) ensuring the connections of the superior middle bases (i.e., spring housing mechanisms) to the most superior planar bases is made so that the double coil torsion spring connection (when untightened) causes the run of the superior middle bases (i.e., spring housing mechanism) to lay perpendicular to the run of the length of the torso so that the squared opening of each superior middle base (i.e., spring housing mechanism) faces the torso,
(D.) ensuring that, for each of the superior-middle (i.e., spring housing mechanism) to superior planar base connection, the superior swing of the superior middle bases (i.e., spring housing mechanisms) allows the hinges to abut against their other halves and that by this move (for each superior-middle and superior planar base connection) the superior middle bases (i.e., spring housing mechanisms) would abut against and run parallel to their corresponding (immediately superior) superior planar bases, when the torsion springs are tightened;
(b.) As described in 13(a.), wherein alternatively there is no flat, rectangular bar to which the compression springs attach; nor hinges; nor double coil torsion springs and the device construction further alters thusly:
(i.) the superior middle bases (i.e., spring housing mechanism) rests against their most superior bases as if the double coil torsion springs were present and maximally engaged--all three couples of bases run parallel to one another, the superior middle resting against the exterior (distal side) of its most immediately superior planar base;
(ii.) the compression springs attach to the superior, squared, axially planar (regarding the body to be tensioned) tops of the inferior middle bases;
(iii.) the compressions springs have guide pins inside their loops;
(iv.) the guide pins attach to the inside side of the axially planar fold of the opening of the superior middle bases, along with the superior ends of each guide pin's associated compression spring;
(v.) the guide pins pass through holes, where the compression springs attach to the inferior middle bases;
(vi.) the inferior portions of the two superior middle bases (i.e., spring housing mechanism) slightly round off, towards the body to be tensioned, in a “u” shape, in a manner that allows for the new “u” opening to slide along the thickness of the inferior of the ultimate superior planar base,
(A.) wherein the inferior of each of the two most superior planar bases fits snugly inside each of these two new “u” shaped opening;
(vii.) the new snug connections are fastened in place;
(viii.) tension springs attach to the inferior of each guide rod (inferiorly past the holes) and the top of these tension springs attaches to the base which has the holes through which the guide rods pass;
(ix.) the general turnbuckle assemblies are replaced by linear tensioning rod assemblies which are constructed as follows:
(A.) starting with two female threaded rods—threading in both length-ending planes of the rod (planes which run perpendicular to the rod's run)—one rod has two female threaded holes and the other has one (the other being a telescoping hole),
(B.) adding two male threaded rods (one as an end rod (attached to its replacements position on the inferior planar base) and the other as just a straight rod with threading with one end attached to the other side of the turnbuckle),
(C.) attaching the other end of the straight rod (threaded) to the side of the other female threaded rod (i.e., the one with the telescoping hole) that threaded,
(D.) telescoping an end rod, wrapped in a compression spring to the telescoping end of the female threaded rod that only has one side female threaded,
(E.) attach that end rod to the inferior middle base.

14. A device according to 7, wherein anywhere along the linear pressure inducing apparatuses or linear pressure inducing apparatus assemblies terminal ends, there are laterally running objects which directly or indirectly distribute the pressure of that induced by the pressure inducing apparatuses or linear pressure inducing apparatus assemblies, whether obliquely run, directly laterally run, or somewhere between those two angles, in general.

15. A device according to claim 10 further comprising one planar base modified such that this planar base is composed of alternating (laterally, longitudinally, or inter-weaving) strips of rigid and pliable (possibly elastic mesh) materials used to displace pressure upon pressure points or help displace center of tensioning pressure.

16. A device according to claim 13 further comprising one planar base modified such that this planar base is composed of alternating (laterally, longitudinally, or inter-weaving) strips of rigid and pliable (possibly elastic mesh) materials used to displace pressure upon pressure points or help displace center of tensioning pressure--e.g., beginning with a right superior planar base that partially lies over the right of a torso's circumference (laterally centered under the right), a left superior planar base that partially lies over the left of a torso's circumference (laterally centered under the left arm pit), and an inferior planar base, which lies around the full circumference of an area that includes the lower abdomen and hips (with a single disunion/opening at the abdomen), for use upon the torso, one attaches, immediately superior the inferior planar base, a spring mechanism made of a pair of crossing scissor rods and a middle base (to which the inferior of turnbuckle assemblies attach—the superior ends attached to the relevant sides superior planar base) on each side of the body; and, above each superior base, a padded, mesh armpit cradle, the totality of the exempli produced as follows:
(a.) wherein two pairs of crossing straight bars (“scissor rods”) engaging new springs (one pair of scissor rods is used upon each side of the body to be tensioned) is places just inferior the inferior middle base on each side of the body beneath one or the other of the superior left planar base or superior right planar base;
(b.) wherein these two crossing scissor rods form an “x” on each side of the body to be tensioned, each between the relevant side's middle (one per side, each middle base being immediately inferior its side's superior left or superior right planar base;
(c.) wherein each pair of crossing scissor rods has four cylindrical rods with a protruding nodule (“small rods”) attached to their four terminal ends;
(d.) wherein (when the cylindrical rods with a protruding nodule are positioned so as to continue the run of its attached larger crossing scissor rod) each nodule protrudes perpendicular to the planar run of its cylindrical rods with a protruding nodule to which each nodule attaches (two nodules obliquely facing one another per superior/inferior diametrically opposed ends of the “x” formed by the crossing scissor rods (two nodules facing one another at the top (superior) and bottom (inferior) of each of the two crossing scissor rods, two facings per side of the body to be tensioned));
(e.) wherein each cylindrical rods with a protruding nodule, of eight, solely connects to one terminal end of one other large crossing scissor rod; and these connections are each a swivel joint connection so that the continuation of the run, just mentioned in “d.”, can bend and swivel—the run of the crossing scissor rod continued by the run of the connected cylindrical rods with a protruding nodule—
(f.) wherein the eight nodules are positioned such that the end of each cylindrical rods with a protruding nodule, which is opposite the swivel joint, runs through a compression spring, which abut against the nodule, the nodule positioned between the two polar ends of the cylindrical rods with a protruding nodule;
(g.) wherein each terminal end (opposite the swivel joints) of the cylindrical rods with a protruding nodule, which passes through compression springs, is designed so as to fit into a prism cap (along with one end of a squared prism rod with a channel on one of its planes (i.e., a channel rod)) without impeding the lateral movement of the small rods beyond that impediment indirectly supplied through the compression springs'resistance;
(h.) wherein the other (non-nodule abutting) ends of these compression springs are each attached to the inside of a unique rectangular prism shaped cap;
(i.) wherein the openings of the caps, into which the springs attach, also have longitudinal space (height) for inserting the squared end of a long square prism rod with a channel along one of its sides (i.e., a channel rod), the squared part of the prism being a little bigger than the nodules'projection's, cylindrical rods with a protruding nodule's and the channel rod's (into which the nodules will insert) combined height;
(j.) wherein each channel (each channel rod) houses two nodules and each end (two terminal ends of the length of the channel rod) of the channel rods is inserted into the extra space of each cap (thus, each pair of crossing scissor rods have a channel rod on two diametrically (inferior/superior) opposed sides: four channel rods in total, one superior and one inferior channel rod per two crossing scissor rods--four caps per side of the body to be tensioned);
(k.) wherein each channel rod is attached to another surface, through hardware which, when used inside the channel, keeps the interior surface of the channels flat, so that the nodules may freely move within the channels;
(l.) wherein this “other surface” is the exterior of the extreme asymptote of a generally hotdog shaped (“u” shaped bar), and one “u” shaped bar is at the top of each crossing scissor rod couple, and one is at the bottom of each crossing scissor rod couple. This creates four channel rod connections to four “u” shaped bars;
(m.) wherein, the middle bases are flat, unfolded or unrounded bases just beneath (centered) each armpit abutting superior planar base;
(n.) wherein the two superior “U” shaped bars (one per pair of crossing scissor rods), each affix to one or the other of the two inferior edges of a middle base so that each inferior edge of a middle base has one superior “U” shaped rod/base attached to it;
(o.) wherein the other two, opposing, “U” shaped bars, at the bottom of the crossing scissor rods, each attach to the superior edge of their side's immediately inferior base (the inferior planar base);
(p.) wherein the superior planar bases are design as they were in 7(a.) and the protrusion of the superior planar bases (where the cut and fold happened), are shifted down the body, with the superior bases to a point at which the fold does not touch the arm pit;
(q.) wherein a new superior base (the hammock base of alternating mesh and solid materials) is formed as follows:
(i.) upon each arm pit there is a flat planar base which runs on the anterior side of the arm and torso (another upon on the posterior side), and between these posterior and anterior bases, within the armpit crease (and a little beyond (inferiorly and distally)) there is a woven mesh of elastic and semi-rigid material which has extra padding on the side that touches the body to be tensioned,
(ii.) connections from the posterior protrusion of that remaining portion of the superior edge of the original superior planar base (remaining from the cut and fold) attach (through many differently angled rods) to the posterior mesh-connected base,
(iii.) connections from the anterior protrusion of that remaining portion of the superior edge of the original superior planar base (remaining from the cut and fold) attach (through many differently angled rods) to the anterior mesh-connected base;
(q.) wherein two sets of two or more turnbuckle assemblies are used on each side of the body and each set is under either the left or right superior planar base, attached from its side superior planar base to its side middle base.

17. A device according to 14, further comprising more than one said planar base on a base level.

18. A device according to 14, further comprising one or more springs (of uniform or varying progressions of thickness) which either, as a collection springs (predominately as a singular linear pressure inducing assembly), induce pressure upon the bases; or, individually or with other springs, mitigate area induced pressure or divert the center of tensioning upon a body to be tensioned.

19. A device according to claim 17 further one or more springs (of uniform or varying progressions of thickness) which either, as a collection springs (predominately as a singular linear pressure inducing assembly), induce pressure upon the bases; or, individually or with other springs, mitigate area induced pressure or divert the center of tensioning upon a body to be tensioned.

20. A device according to claim 19, further comprising materials used to displace pressure upon pressure points or help displace center of tensioning pressure.
¹ Whereas the author could not find the very rare use of the ablative plural of gratia in the Black's Law Dictionary, the very rare situation seen in claim 4 grammatically requires the ablative plural “gratiīs”, instead of the ablative singular gratia, due to the nature of the interaction of the genitive singular example/exempli (the non-exhaustive list) conferring multiple benefits, due to the manner in which the broad category of “spring” and the exempli of uses calls back to the exempli of specific spring types, in which many times, multiple spring types are referenced and thus multiple levels of benefits and cross-overs are intended to be specified here.
